(19) United States
(12) Patent Application Publication
(10) Pub. No.: US 2022/0177835 A1
(43) Pub. Date: Jun. 9, 2022

(54) METHODS OF GENERATING AND ISOLATING MIDBRAIN DOPAMINE NEURONS

(71) Applicant: MEMORIAL SLOAN-KETTERING CANCER CENTER, New York, NY (US)

(72) Inventors: Lorenz Studer, New York, NY (US); Taewan Kim, New York, NY (US); Stefan Irion, New York, NY (US); So Yeon Koo, New York, NY (US)

(73) Assignee: MEMORIAL SLOAN-KETTERING CANCER CENTER, New York, NY (US)

(21) Appl. No.: 17/681,113

(22) Filed: Feb. 25, 2022

Related U.S. Application Data
(63) Continuation of application No. PCT/US2020/048733, filed on Aug. 31, 2020.
(60) Provisional application No. 62/893,674, filed on Aug. 29, 2019.

(51) Int. Cl. C12N 5/0793; A61K 35/30

(52) U.S. Cl. CPC C12N 5/0619; A61K 35/30; C12N 2501/119; C12N 2501/15; C12N 2506/02; C12N 2501/41; C12N 2501/415; C12N 2506/45; C12N 2501/16

(57) ABSTRACT

The present disclosure provides methods for generating midbrain dopamine neurons (mDAs) and precursors thereof, mDAs and precursors thereof generated by such methods and compositions comprising such cells, and uses thereof for preventing and/or treating neurological disorders. The present disclosure further provides methods of isolating mDAs and precursors thereof from a cell population using novel surface markers.

### CROSS REFERENCE TO RELATED APPLICATIONS

The present application is a Continuation of International Patent Application No. PCT/US2020/048733, filed Aug. 31, 2020, which claims priority to U.S. Provisional Application No. 62/893,674, filed Aug. 29, 2019, to each of which priority is claimed and the contents of each of which are incorporated herein by reference in their entireties.

### 1. INTRODUCTION

The present disclosure provides methods for generating midbrain dopamine neurons (mDAs) and precursors thereof, mDAs and precursors thereof generated by such methods and composition comprising such cells. The present disclosure also provides uses of the mDAs and composition comprising thereof for preventing and/or treating neurological disorders. The present disclosure further provides methods of isolating mDAs and precursors thereof from a cell population using novel surface markers.

### 2. BACKGROUND

Previously, embryonic and somatic stem cells were used as therapeutics and model systems for neurodegenerative diseases. Research and technological developments relating to directed differentiation of embryonic and somatic stem cells has taken place in the field of diseases of the central nervous system (CNS), such as for Huntington's, Alzheimer's, Parkinson's, and multiple sclerosis. However the results of these studies showed little in vivo capability to restore neuronal function and often resulted in the growth of unwanted tumors in the patients.

Therefore, there is still a need for improved methods for generating midbrain dopamine neurons that are suitable for treating neurodegenerative disorders such as Parkinson's disease.

### 3. SUMMARY OF THE INVENTION

The present disclosure provides methods for generating midbrain dopamine neurons (mDAs) and precursors thereof, mDAs and precursors thereof generated by such methods, compositions comprising such cells, and uses of such cells and compositions for preventing and/or treating neurological disorders. In addition, the present disclosure provides methods of isolating mDAs and precursors thereof from a cell population using novel surface markers.

In certain embodiments, the present disclosure provides an in vitro method for inducing differentiation of stem cells, comprising: contacting the stem cells with at least one inhibitor of Small Mothers Against Decapentaplegic (SMAD) signaling, at least one activator of Sonic hedgehog (SHH) signaling, and at least one activator of wingless (Wnt) signaling; and contacting the cells with at least one activator of fibroblast growth factor (FGF) signaling to obtain a population of differentiated cells expressing at least one marker indicating a midbrain dopamine neuron (mDA) or a precursor thereof, wherein the at least one activator of FGF signaling is selected from FGF18, FGF17, FGF8a, and combination thereof.

In certain embodiments, the present disclosure provides an in vitro method for inducing differentiation of stem cells, comprising: contacting the stem cells with at least one inhibitor of Small Mothers Against Decapentaplegic (SMAD) signaling, at least one activator of Sonic hedgehog (SHH) signaling, and at least one activator of wingless (Wnt) signaling; and contacting the cells with at least one activator of fibroblast growth factor (FGF) signaling to obtain a population of differentiated cells expressing at least one marker indicating a midbrain dopamine neuron (mDA) or a precursor thereof, wherein the initial contact of the cells with the at least one activator of FGF signaling is at least about 5 days from the initial contact of the cells with the at least one inhibitor of SMAD signaling.

In certain embodiments, the cells are contacted with the at least one activator of FGF signaling for at least about 1 day. In certain embodiments, the cells are contacted with the at least one activator of FGF signaling for up to about 15 days. In certain embodiments, the cells are contacted with the at least one activator of FGF signaling for about 5 days.

In certain embodiments, the initial contact of the cells with the at least one activator of FGF signaling is at least about 5 days from the initial contact of the cells with the at least one inhibitor of SMAD signaling.

In certain embodiments, the initial contact of the cells with the at least one activator of FGF signaling is about 10 days from the initial contact of the cells with the at least one inhibitor of SMAD signaling. In certain embodiments, the initial contact of the cells with the at least one activator of FGF signaling is 12 days from the initial contact of the cells with the at least one inhibitor of SMAD signaling.

In certain embodiments, the cells are contacted with the at least one inhibitor of SMAD signaling for about 5 days. In certain embodiments, the cells are contacted with the at least one inhibitor of SMAD signaling for 7 days.

In certain embodiments, the cells are contacted with the at least one activator of SHH signaling for about 5 days. In certain embodiments, the cells are contacted with the at least one activator of SHH signaling for 7 days.

In certain embodiments, the cells are contacted with the at least one activator of Wnt signaling for about 10 days. In certain embodiments, the cells are contacted with the at least one activator of Wnt signaling for 12 days.

In certain embodiments, the concentration of the at least one activator of Wnt signaling is increased about 4 days from its initial contact with the stem cells. In certain embodiments, the concentration of the at least one activator of Wnt signaling is increased by between about 300% and about 1000% from the initial concentration of the at least one activator of Wnt signaling. In certain embodiments, the concentration of the at least one activator of Wnt signaling is increased to a concentration of between about 3 μM and 10 μM. In certain embodiments, the concentration of the at least one activator of Wnt signaling is increased to a concentration of about 3 μM. In certain embodiments, the concentration of the at least one activator of Wnt signaling is increased to a concentration of about 7.5 μM.

In certain embodiments, the at least one activator of FGF signaling comprises FGF18.

In certain embodiments, the at least one inhibitor of SMAD signaling is selected from inhibitors of TGFβ/Activin-Nodal signaling, inhibitors of bone morphogenetic protein (BMP) signaling, and combinations thereof.

In certain embodiments, the at least one inhibitor of TGFβ/Activin-Nodal signaling is comprises an inhibitor of ALK5.

In certain embodiments, the at least one inhibitor of TGFβ/Activin-Nodal signaling comprises SB431542, or a derivative, or a mixture thereof. In certain embodiments, the derivative of SB431542 is A83-01. In certain embodiments, the at least one inhibitor of TGFβ/Activin-Nodal signaling comprises SB431542.

In certain embodiments, the at least one inhibitor of BMP signaling comprises LDN193189, Noggin, dorsomorphin, a derivative thereof, or a mixture thereof. In certain embodiments, the at least one inhibitor of BMP comprises LDN-193189.

In certain embodiments, the at least one activator of Wnt signaling comprises an inhibitor of glycogen synthase kinase 3β (GSK3β) signaling.

In certain embodiments, the at least one activator of Wnt signaling is selected from CHIR99021, BIO, CHIR98014, Lithium, 3F8, Wnt3A, Wnt1, Wnt5a, derivatives thereof, and mixtures thereof. In certain embodiments, the at least one activator of Wnt signaling comprises CHIR99021.

In certain embodiments, the at least one activator of SHH signaling is selected from SHH protein, Smoothened agonists (SAG), derivatives thereof, and mixtures thereof. In certain embodiments, the SHH protein comprises a recombinant SHH, a purified SHH, or a combination of the foregoing.

In certain embodiments, the recombinant SHH comprises a recombinant protein that is at least about 80% identical to a mouse Sonic Hedgehog N-terminal fragment. In certain embodiments, the recombinant SHH comprises SHH C25II. In certain embodiments, the SAG comprises purmorphamine.

In certain embodiments, the at least one marker indicating a midbrain dopamine neuron or a precursor thereof is selected from EN1, OTX2, TH, NURR1, FOXA2, PITX3, LMX1A, LMO3, SNCA, ADCAP1, CHRNA4, GIRK2, and combinations thereof.

In certain embodiments, the differentiated cells have a detectable level of expression of the at least one marker indicating a midbrain dopamine neuron or a precursor thereof at least about 10 days from the initial contact of the stem cells with the at least one inhibitor of SMAD signaling.

In certain embodiments, the differentiated cells have a detectable level of expression of EN1 about 30 days from the initial contact of the stem cells with the at least one inhibitor of SMAD signaling. In certain embodiments, the differentiated cells have a detectable level of expression of EN1 about 40 days from the initial contact of the stem cells with the at least one inhibitor of SMAD signaling.

In certain embodiments, the differentiated cells do not express at least one marker selected from PAX6, EMX2, LHX2, SMA, SIX1, PITX2, SIM1, POU4F1, PHOX2A, BARHL1, BARHL2, GBX2, HOXA2, HOXB2, POUSF1, NANOG, and combinations thereof.

In certain embodiments, the method further comprises subjecting the population of differentiated cells to conditions favoring differentiation of midbrain dopamine neuron precursors to midbrain dopamine neurons.

In certain embodiments, the conditions comprise exposing the cells to at least one of brain-derived neurotrophic factor (BDNF), glial cell-derived neurotrophic factor (GDNF), Cyclic adenosine monophosphate (cAMP), Transforming growth factor beta 3 (TGFP3), ascorbic acid (AA), and DAPT.

In certain embodiments, the stem cells are selected from human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells. In certain embodiments, the stem cells are human stem cells. In certain embodiments, the stem cells are pluripotent or multipotent stem cells. In certain embodiments, the stem cells are pluripotent stem cells. In certain embodiments, the pluripotent stem cells are selected from embryonic stem cells, induced pluripotent stem cells, and combinations thereof.

In another aspect, the present disclosure provides a cell population of in vitro differentiated cells, wherein said in vitro differentiated cells are obtained by any preceding methods.

In another aspect, the present disclosure provides a cell population of in vitro differentiated cells, wherein at least about 50% of the cells express at least one marker indicating a midbrain dopamine neuron or a precursor thereof, and less than about 50% of the differentiated cells express at least one marker selected from PAX6, EMX2, LHX2, SMA, SIX1, PITX2, SIM1, POU4F1, PHOX2A, BARHL1, BARHL2, GBX2, HOXA2, HOXB2, POUSF1, NANOG, and combinations thereof. In certain embodiments, the at least one marker indicating a midbrain dopamine neuron or a precursor thereof is selected from EN1, OTX2, TH, NURR1, FOXA2, LMX1A, PITX3, LMO3, SNCA, ADCAP1, CHRNA4, GIRK2.

In another aspect, the present disclosure provides a composition comprising the cell population disclosed herein. In certain embodiments, the composition is a pharmaceutical composition further comprising a pharmaceutically acceptable carrier.

In another aspect, the present disclosure provides a method for isolating midbrain dopamine neurons and precursors thereof from a population of cells, comprising isolating cells that do not express a detectable level of at least one negative surface marker and express a detectable level of at least one positive surface marker.

In another aspect, the present disclosure provides a method for isolating midbrain dopamine neurons and precursors thereof from a population of cells, comprising isolating cells that (a) do not express a detectable level or express a reduced level of at least one negative surface marker as compared to the mean expression level of the at least one negative surface marker in the population of cells; and (b) an increased level of at least one positive surface marker as compared to the mean expression level of the at least one positive marker in the population of cells.

In certain embodiments, the at least one positive surface marker is selected from CD171, CD184, and combinations thereof. In certain embodiments, the at least one positive surface marker comprises CD184. In certain embodiments, the at least one negative surface marker is selected from CD49e, CD99, CD340, and combinations thereof. In certain embodiments, the at least one negative surface marker comprises CD49e. In certain embodiments, the method comprises isolating cells that do not express a detectable level of CD49e and express a detectable level of CD184.

In certain embodiments, the method comprises isolating cells that do not express a detectable level or express a reduced level of CD49e as compared the mean expression level of CD49e in the population of cells; and express an increased level of CD184 as compared to the mean expression level of CD184 in the population of cells.

In another aspect, the present disclosure provides a cell population of in vitro differentiated cells, wherein at least about 50% of the cells express a detectable level of at least one positive surface marker and do not express a detectable level of at least one negative surface marker.

In another aspect, the present disclosure provides a cell population of in vitro differentiated cells, wherein at least about 50% of the cells express an increased level of at least one positive surface marker as compared to the mean expression level of the at least one positive marker in the population of cells; and do not express a detectable level or express a reduced level of at least one negative surface marker as compared to the mean expression level of the at least one negative surface marker in the population of cells.

In certain embodiments, the at least one positive surface marker is selected from CD171, CD184, and combinations thereof. In certain embodiments, the at least one positive surface marker comprises CD184. In certain embodiments, the at least one negative surface marker is selected from CD49e, CD99, CD340, and combinations thereof. In certain embodiments, the at least one negative surface marker comprises CD49e. In certain embodiments, the at least one positive surface marker comprises CD184 and the at least one negative surface marker comprises CD49e.

In another aspect, the present disclosure provides a composition comprising the cell population disclosed herein. In certain embodiments, the composition disclosed herein is a pharmaceutical composition further comprising a pharmaceutically acceptable carrier. In another aspect, the present disclosure provides kit for inducing differentiation of stem cells to midbrain dopamine neurons or precursors thereof, comprising: (a) at least one inhibitor of SMAD signaling; (b) at least one activator of SHH signaling; (c) at least one activator of Wnt signaling; and (d) at least one activator of FGF signaling.

In certain embodiments, the kit further comprises (f) instructions for inducing differentiation of the stem cells into a population of differentiated cells that express at least one midbrain DA precursor marker.

In another aspect, the present disclosure provides a method of preventing and/or treating a neurodegenerative disorder in a subject, comprising administering to the subject an effective amount of one of the followings: (a) the cell population disclosed herein; or (b) the composition disclosed herein.

In certain embodiments, the neurodegenerative disorder is Parkinson's disease, Huntington's disease, Alzheimer's disease, or multiple sclerosis.

In certain embodiments, the cell population disclosed herein or the composition disclosed herein is for use in preventing and/or treating a neurodegenerative disorder in a subject. In certain embodiments, the neurodegenerative disorder is Parkinson's disease, Huntington's disease, Alzheimer's disease, or multiple sclerosis.

### 4. BRIEF DESCRIPTION OF THE DRAWINGS

FIG. 1 shows EN1 expression in stem-cell-derived mDAs and precursors from day 3 to day 30 using the Wnt-Boost protocol.

FIG. 2 depicts the protocol using FGF8b in combinations with the Wnt-Boost protocol.

FIG. 3 shows that EN1 protein was maintained in the differentiated cells in a FGF8b contacting duration dependent manner. Cells expressing EN1 also expressed FOXA2 and LMX1A.

FIGS. 4A-4B show mRNA expression levels of the differentiated cells measured by qRT-PCR on day 30. FIG. 4A shows mRNA expression level of EN1 was maintained in contacting duration dependent manner of FGF8b, and the expression levels of FOXA2, NURR1, and TH were comparable in all conditions. FIG. 4B shows mRNA expression levels of non-mDA markers, e.g., SMA and SIX1, which were induced in a FGF8b contacting duration dependent manner.

FIG. 5 shows SIX1 immuno-staining of FGF8b treated cells on day 30 of differentiation.

FIG. 6 shows mRNA expressions of markers in FGFs treated cells on day 16 of differentiation.

FIG. 7 shows immunostaining of markers in FGF8b and FGF18 treated cells on day 16 of differentiation.

FIG. 8 shows RNA expressions of markers in FGF8b and FGF18 treated cells on day 27 and day 40 of differentiation.

FIG. 9A is a schematic showing of a donor vector structure for NURR1::GFP reporter hPSCs.

FIG. 9B shows NURR1 mRNA level in cells differentiated from hPSCs using the Wnt-Boost protocol.

FIG. 9C shows FACS results of midbrain DA neurons differentiated from H9-hPSC and NURR1::GFP hPSCs on day 25 of differentiation.

FIG. 10A shows single cell qRT-PCR results in NURR1:GFP positive cells isolated on day 25 and day 40 of differentiation.

FIG. 10B shows immune-staining of NURR1 sorted mDAs expressing TH, FOXA2, and NURR1 on day 60 of differentiation. Cells were sorted on day 25 followed by continuous culturing until day 60.

FIG. 11 provides immune-staining images of the in vivo grafted NURR1::GFP positive midbrain DA neurons 8 weeks after the injection of the cells to the immune-deficient mice.

FIG. 12 provides immune-staining images of NURR1:GFP positive cells cultured under the WNT-Boost protocol and the WNT-boost+FGF18 (day 12-day 16) protocol. Cells were sorted on day 25 followed by continuous culturing until day 40.

FIG. 13A provides FOXA2, EN1, and NURR1 mRNA expressions in NURR1:GFP positive cells cultured under the WNT-Boost protocol and the WNT-boost+FGF18 (day 12-day 16) protocol. mRNA expression in both sorted and non-sorted cells were compared.

FIG. 13B shows sorted NURR1:GFP positive cells cultured under the WNT-Boost protocol and the WNT-boost+FGF18 (day 12-day 16) protocol were grafted to immune-deficient mice. Neurite outgrowth and TH and SC121 expression in the grated cells were detected.

FIG. 14 shows 390 surface markers were screened in mDAs on day 25 of differentiation derived from NURR1::GFP hPSC using the Wnt-Boost protocol. Antibodies were conjugated to PE, FITC, or APC.

FIG. 15A shows that positive surface markers CD171 and CD184 were enriched in NURR1⁺ cells.

FIG. 15B provides the RNA expressions of CD171 and CD184 in differentiated cells using the Wnt-Boost protocol.

FIG. 16A shows that negative surface markers CD49e, CD99 and CD340 were enriched in NURR1+ cells.

FIG. 16B provides the RNA expressions of CD49e, CD99 and CD340 in differentiated cells using the Wnt-Boost protocol.

FIG. 17 provides morphology of cells sorted by CD49e weak or CD49e high. Cells were sorted on day 25 of in vitro differentiation under WNT-boost and WNT-boost+FGF18 protocols. After sorting, cells were then cultured for another 15 days.

FIG. 18 shows immuno-staining images of sorted CD49e weak cells on day 40 of in vitro differentiation under the WNT-Boost protocol and the WNT-boost+FGF18 protocol.

FIG. 19 shows FACS results for sorting of CD49e-based purification of mDAs derived from the hPSC cell line MEL1.

FIG. 20 shows morphology of MEL1 hPSC derived mDA cells sorted by CD49e weak or CD49e high. Cells were sorted on day 25 of in vitro differentiation under the WNT-Boost protocol and the WNT-boost+FGF18 protocol. After sorting, cells were then cultured for another 15 days.

FIG. 21 shows immuno-staining images of MEL1 hPSC derived CD49e weak mDAs on day 40 after in vitro differentiation under the WNT-Boost protocol and the WNT-boost+FGF18 protocol. CD49e weak cells were sorted on day 25.

FIG. 22 shows the relative mRNA expression in MEL1 hPSC derived CD49e weak mDAs on day 40 after in vitro differentiation under the WNT-boost protocol and the WNT-boost+FGF18 protocol. CD49e weak cells were sorted on day 25.

FIG. 23 shows morphology of CD49e, CD99, or CD340 sorted cells on day 40 of in vitro differentiation under the WNT-Boost+FGF18 protocol. Cells were sorted on day 25 of the in vitro differentiation.

FIG. 24 shows the relative mRNA expression of CD49e, CD99, or CD340 sorted cells on day 40 of in vitro differentiation under the WNT-Boost+FGF18 protocol. Cells were sorted on day 25 of the in vitro differentiation.

FIG. 25 shows FACS sorting results of NURR1+ cells sorted by 49E (PE) and 171 (APC) on day 25 of in vitro differentiation under the WNT-Boost protocol.

FIG. 26 shows FACS sorting results of NURR1+ cells sorted by 49E (PE) and 184 (APC) on day 25 of in vitro differentiation under the WNT-Boost protocol.

FIG. 27 provides morphology of cells sorted by CD49e, CD171, CD188. Cells were sorted on day 25 of in vitro differentiation under the WNT-Boost protocol. After sorting, cells were then cultured for another 2 days.

FIG. 28 shows mRNA expression of cells sorted by CD49e, CD171, CD188. Cells were sorted on day 25 of in vitro differentiation under the WNT-Boost protocol. After sorting, cells were then cultured for another 2 days.

FIG. 29 shows enrichment of the NURR1::GFP population in single CD49e weak sorted cells at day 25 of in vitro differentiation.

FIG. 30 shows enrichment of the NURR1:GFP population in CD49e weak CD184 high double sorted cells at day 25 of in vitro differentiation.

FIG. 31 shows that TH midbrain DA neurons co-expressed with FOXA2 and GFP, implying midbrain DA neuron identity at the enriched NURR1:GFP population of FIG. 30.

FIG. 32 shows midbrain DA marker mRNA expressions in cells sorted by CD 49e and CD 184 on day 25 of in vitro differentiation. After sorting, cells were cultured for another 15 days in vitro.

FIG. 33 shows non-midbrain DA mRNA expressions in cells sorted by CD 49e and CD 184 on day 25 of in vitro differentiation. After sorting, cells were cultured for another 10 days in vitro.

FIGS. 34A-34D show the in vivo survival of transplanted cells sorted with presently disclosed CD markers (CD49e depleted and CD184 enriched) after in vitro differentiation under the WNT-Boost protocol. FIG. 34A shows robust survival of the sorted cells and enrichment of TH⁺ cells within the graft as compared to unsorted cells. FIG. 34B shows reduced number of SOX2⁺ precursors and KI67⁺ dividing cells one month after grafting. FIG. 34C shows quantification of SOX2⁺ staining cells in FIG. 34B. FIG. 34D shows quantification of Ki67⁺ cells in FIG. 34B.

FIGS. 35A-35B show the in vivo survival and EN1 expression of cells differentiated under the WNT-boost and WNT-boost+FGF18 protocols. FIG. 35A shows the percentage of cells expressing EN1. FIG. 35B shows the emerging of striatal innervation with fibers at the transplantation sites.

### 5. DETAILED DESCRIPTION

The present disclosure provides methods for generating mDAs and precursors thereof, mDAs and precursors thereof generated by such methods, compositions comprising such cells, and uses thereof for preventing and/or treating neurological disorders. In addition, the present disclosure provides methods of isolating mDAs and precursors thereof from a cell population using novel surface markers.

The present disclosure is at least based on the discovery that stem-cell derived mDAs generated by the presently disclosed methods have sustained expression of EN1, e.g., the expression of EN1 is maintained throughout the development and maturation of mDAs.

Non-limiting embodiments of the presently disclosed subject matter are described by the present specification and Examples.

For purposes of clarity of disclosure and not by way of limitation, the detailed description is divided into the following subsections:
5.1. Definitions;
5.2. Methods of Differentiating Stem Cells;
5.3. Methods of Isolating Midbrain DA Neurons and Precursor Thereof,
5.4. Compositions Comprising Midbrain DA Neurons and Precursors Thereof,
5.5. Methods of Treating Neurodegenerative Disorders; and
5.6. Kits.

### 5.1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, a wingless (Wnt) complex protein, including beta-catenin, NOTCH, transforming growth factor beta (TGFP), Activin, Nodal, glycogen synthase kinase 3β (GSK3β) proteins, bone morphogenetic proteins (BMP) and fibroblast growth factors (FGF). For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TFGP), Activin, Nodal, bone morphogenic proteins (BMPs), etc.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, a named associated molecule, such as a glycogen synthase kinase 3β (GSK3β)) (e.g., including, but not limited to, the signaling molecules described herein), for one example, via directly contacting SMAD signaling, contacting SMAD mRNA, causing conformational changes of SMAD, decreasing SMAD protein levels, or interfering with SMAD interactions with signaling partners (e.g., including those described herein), and affecting the expression of SMAD target genes (e.g. those described herein).

Inhibitors also include molecules that indirectly regulate biological activity, for example, SMAD biological activity, by intercepting upstream signaling molecules (e.g., within the extracellular domain, examples of a signaling molecule and an effect include: Noggin which sequesters bone morphogenic proteins, inhibiting activation of ALK receptors 1,2,3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules). Antibodies that block upstream or downstream proteins are contemplated for use to neutralize extracellular activators of protein signaling, and the like. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators,” as used herein, refer to compounds that increase, induce, stimulate, activate, facilitate, or enhance activation the signaling function of the molecule or pathway, e.g., Wnt signaling, SHH signaling, etc.

As used herein, the term “WNT” or “wingless” in reference to a ligand refers to a group of secreted proteins (e.g., integration 1 in humans) that are capable of interacting with a WNT receptor, such as a receptor in the Frizzled and LRPDerailed/RYK receptor family.

As used herein, the term “a WNT or wingless signaling pathway refers to a signaling pathway composed of Wnt family ligands and Wnt family receptors, such as Frizzled and LRPDerailed/RYK receptors, mediated with or without β-catenin. In certain embodiments, the WNT signaling pathway include mediation by β-catenin, e.g., WNT/-catenin.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of midbrain DA precursors, or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation.

As used herein, the term “neuron” refers to a nerve cell, the principal functional units of the nervous system. A neuron consists of a cell body and its processes—an axon and at least one dendrite. Neurons transmit information to other neurons or cells by releasing neurotransmitters at synapses.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as neural, neural crest, cranial placode, and non-neural ectoderm precursors. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein marker of mDAs or precursors thereof, such as EN1, OTX2, TH, NURR1, FOXA2, LMX1A, PITX3, LMO3, SNCA, ADCAP1, CHRNA4, and GIRK2).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “negative”, “weak”, or “−” when used in reference to any surface marker disclosed herein refer to that the surface marker (e.g., CD49e) is not expressed at a detectable level, or is expressed at a reduced level in a cell as compared to the mean expression of the surface marker in a population of cells of which the cell is selected or sorted from. As used herein, the term “high”, “strong”, “+”, or “positive” when used in reference to any surface marker disclosed herein refer to that the surface marker (e.g., CD184) is expressed at a detectable level or expressed at an increased level as compared to the mean expression of the surface marker in a population of cells.

In certain embodiments, the cells are distinguished according to their surface marker expression levels based on a readily discernible differences in staining intensity as is known to one or ordinary skill in the art. In certain embodiments, the cut off for designating a cell as a surface marker “weak”, “negative”, or “−” cell can be set in terms of the staining intensity distribution (e.g., fluorescence intensity distribution) observed for all the cells, with those cells falling below about 50%, about 40%, about 30%, about 20%, about 10%, or about 5% of staining intensity being designated as the surface marker “weak”, “negative”, or “−” cell. In certain embodiments, the cut off for designating a cell as a surface marker “strong”, “high”, “+”, or “positive” cell can be set in terms of the staining intensity distribution (e.g., fluorescence intensity distribution) observed for all the cells, with those cells falling above about 50%, about 60%, about 70%, about 80%, about 90%, or about 95% of staining intensity being designated as the surface marker “strong”, “high”, “+”, or “positive” cell. In certain embodiments, the frequency distribution of the surface marker staining is obtained for all the cells and the population curve fit to a higher staining and lower staining population, and cells assigned to the population to which they most statistically are likely to belong in view of a statistical analysis of the respective population distributions.

### 5.2. Method of Differentiating Stem Cells

The present disclosure provides methods for inducing differentiation of stem cells, comprising contacting stem cells with at least one inhibitor of Small Mothers Against Decapentaplegic (SMAD) signaling (referred to as “SMAD inhibitor”), at least one activator of Sonic hedgehog (SHH) signaling (referred to as “SHH activator”), and at least one activator of wingless (Wnt) signaling (referred to as “Wnt activator”); and further contacting the cells with at least one activator of fibroblast growth factor (FGF) signaling (referred to as “FGF activator”), to obtain a cell population comprising differentiated cells expressing at least one marker indicating a mDA or a mDA precursor.

In certain embodiments, the at least one FGF activator is capable of promoting midbrain development. In certain embodiments, the at least one FGF activator is selected from FGF8a, FGF17, FGF18, FGF2, and FGF4. In certain embodiments, the at least one FGF activator is selected from FGF8a, FGF17, and FGF18. In certain embodiments, the at least one FGF activator comprises FGF18.

In certain embodiments, the initial contact of the cells with the at least one activator of FGF signaling is at least about 5 days from the initial contact of the cells with the at least one inhibitor of SMAD signaling. In certain embodiments, the initial exposure of the cells to the at least one FGF activator is at least about 10 days from initial exposure of the stem cells to the at least one SMAD inhibitor. In certain embodiments, the FGF activator is selected from FGF8a, FGF17, FGF18, FGF8b, FGF2, and FGF4. The exposure of the cells to the at least one FGF activator prolongs the expression of EN1 by the differentiated cells.

In certain embodiments, the at least one marker indicating a mDA or a mDA precursor is selected from EN1, FOX1A, LMX1A, OTX2, NURR1, TH, PITX3, LMO3, SNCA, ADCAP1, CHRNA4, and GIRK2.

In certain embodiments, the concentration of the at least one Wnt activator is increased during its exposure to the cells. In certain embodiments, said increase of the concentration of the at least one Wnt activator is initiated about 4 days from initial exposure of the stem cells to the at least one SMAD inhibitor. In certain embodiments, the concentration of the at least one Wnt activator is increased by about 300% to about 1000%.

In certain embodiments, the cells are exposed to the at least one Wnt activator with the increased concentration for at least about 7 days. In certain embodiments, at least one additional Wnt activator is added to increase the overall concentration of the Wnt activator(s).

In certain embodiments, the methods further comprise contacting the cells with midbrain DA lineage specific activators and inhibitors, for example, BDNF, GDNF, cAMP, TGFβ, ascorbic acid (AA), and/or DAPT, to induce differentiation of mDA precursors to mDAs.

### 5.2.1. Stem Cells

The presently disclosed subject matter provides in vitro methods for inducing differentiation of stem cells to produce mDAs and precursors thereof. In certain embodiments, the stem cells are pluripotent stem cells. In certain embodiments, the pluripotent stem cells are selected from embryonic stem cells (ESCs), induced pluripotent stem cells (iPSCs), and combinations thereof. In certain embodiments, the stem cells are multipotent stem cells. Non-limiting examples of stem cells that can be used with the presently disclosed methods include human, nonhuman primate or rodent nonembryonic stem cells, embryonic stem cells, induced nonembryonic pluripotent cells and engineered pluripotent cells. In certain embodiments, the stem cells are human stem cells. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell is a human embryonic stem cell (hESC). In certain embodiments, the stem cell is a human induced pluripotent stem cell (hiPSC).

In certain embodiments, the stem cell or a progeny cell thereof contains an introduced heterologous nucleic acid, where said nucleic acid may encode a desired nucleic acid or protein product or have informational value (see, for example, U.S. Pat. No. 6,312,911, which is incorporated by reference in its entirety). Non-limiting examples of protein products include markers detectable via in vivo imaging studies, for example receptors or other cell membrane proteins. Non-limiting examples of markers include fluorescent proteins (such as green fluorescent protein (GFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet, EYFP)), β-galactosidase (LacZ), chloramphenicol acetyltransferase (cat), neomycin phosphotransferase (neo), enzymes (such as oxidases and peroxidases), and antigenic molecules. As used herein, the terms “reporter gene” or “reporter construct” refer to genetic constructs comprising a nucleic acid encoding a protein that is easily detectable or easily assayable, such as a colored protein, fluorescent protein such as GFP or an enzyme such as beta-galactosidase (lacZ gene). In certain embodiments, the reporter can be driven by a recombinant promoter of a premature post-mitotic midbrain DA neuron marker gene, for example, NURR1.

### 5.2.2. SMAD Inhibitors

Non-limiting examples of SMAD inhibitors include inhibitors of transforming growth factor beta (TGFβ)/Activin-Nodal signaling (referred to as “TGFβ/Activin-Nodal inhibitor”), and inhibitors of bone morphogenetic proteins (BMP) signaling. In certain embodiments, the TGFβ/Activin-Nodal inhibitor can neutralize the ligands including TGFβs, BMPs, Nodal, and activins, and/or block their signal pathways through blocking the receptors and downstream effectors. Non-limiting examples of TGFβ/Activin-Nodal inhibitors include those disclosed in WO/2010/096496, WO/2011/149762, WO/2013/067362, WO/2014/176606, WO/2015/077648, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20 (2012), all of which are incorporated by reference in their entireties herein for all purposes. In certain embodiments, the at least one TGFβ/Activin-Nodal inhibitor is selected from inhibitors of ALK5, inhibitors of ALK4, inhibitors of ALK7, and combinations thereof). In certain embodiments, the TGFβ/Activin-Nodal inhibitor comprises an inhibitor of ALK5. In certain embodiments, the TGFβ/Activin-Nodal inhibitor is a small molecule selected from SB431542, derivatives thereof, and mixtures thereof. “SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

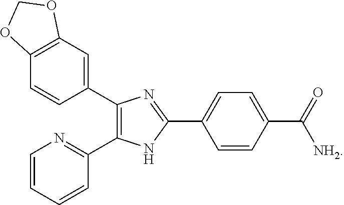 [SMILES: NC(=O)c1ccc(-c2nc(-c3ccc4c(c3)OCO4)c(-c3ccccn3)[nH]2)cc1]

In certain embodiments, the TGFβ/Activin-Nodal inhibitor comprises SB431542. In certain embodiments, the TGFβ/Activin-Nodal inhibitor comprises a derivative of SB431542. In certain embodiments, the derivative of SB431542 is A83-01.

In certain embodiments, the at least one SMAD inhibitor comprises an inhibitor of BMP signaling (referred to as “BMP inhibitor”). Non-limiting examples of BMP inhibitors include those disclosed in WO2011/149762, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20, all of which are incorporated by reference in their entireties. In certain embodiments, the BMP inhibitor is a small molecule selected from LDN193189, Noggin, dorsomorphin, derivatives thereof, and mixtures thereof. “LDN193189” refers to a small molecule DM-3189, IUPAC name 4-(6-(4-(piperazin-1-yl)phenyl)pyrazolo[1,5-a]pyrimidin-3-yl)quinoline, with a chemical formula of C₂₅H₂₂N₆ with the following formula.

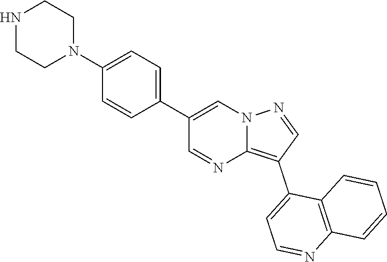 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18: 4388-4392, herein incorporated by reference).

In certain embodiments, the BMP inhibitor comprises LDN193189. In certain embodiments, the BMP inhibitor comprises Noggin.

In certain embodiments, the stem cells are exposed to one SMAD inhibitor, e.g., one TGFβ/Activin-Nodal inhibitor. In certain embodiments, the one TGFβ/Activin-Nodal inhibitor is SB431542 or A83-01. In certain embodiments, the stem cells are exposed to two SMAD inhibitors. In certain embodiments, the two SMAD inhibitors are a TGFβ/Activin-Nodal inhibitor and a BMP inhibitor. In certain embodiments, the stem cells are exposed to SB431542 or A83-01, and LDN193189 or Noggin. In certain embodiments, the stem cells are exposed to SB431542 and LDN193189.

In certain embodiments, the stem cells are exposed to or contacted with at least one SMAD inhibitor for at least about 5 days, or at least about 10 days. In certain embodiments, the stem cells are contacted with or exposed to the at least one SMAD inhibitor for up to about 5 days, or up to about 10 days. In certain embodiments, the stem cells are contacted with or exposed to the at least one SMAD inhibitor for between about 5 days and about 10 days. In certain embodiments, the stem cells are contacted with or exposed to the at least one SMAD inhibitor for about 5 days. In certain embodiments, the stem cells are contacted with or exposed to the at least one SMAD inhibitor for 7 days. In certain embodiments, the cells are contacted with or exposed to the at least one SMAD inhibitor from day 0 through day 6. In certain embodiments, the at least one SMAD inhibitor is added every day or every other day to a cell culture medium comprising the stem cells from day 0 through day 6. In certain embodiments, the at least one SMAD inhibitor is added every day (daily) to a cell culture medium comprising the stem cells from day 0 to day 6.

In certain embodiments, the cells are contacted with or exposed to a TGFβ/Activin-Nodal inhibitor. In certain embodiments, the concentration of the TGFβ/Activin-Nodal inhibitor contacted with or exposed to the cells is between about 1 μM and about 20 μM, between about 1 μM and about 10 μM, between about 1 μM and about 15 μM, between about 10 μM and about 15 μM, between about 5 μM and about 10 μM, between about 5 μM and about 15 μM, between about 5 μM and about 20 μM, or between about 15 μM and about 20 μM. In certain embodiments, the concentration of the TGFβ/Activin-Nodal inhibitor contacted with or exposed to the cells is between about 1 μM and about 10 μM. In certain embodiments, the concentration of the TGFβ/Activin-Nodal inhibitor contacted with or exposed to the cells is about 5 μM. about 10 μM. In certain embodiments, the concentration of the TGFβ/Activin-Nodal inhibitor contacted with or exposed to the cells is about 10 μM. In certain embodiments, the TGFβ/Activin-Nodal inhibitor comprises SB431542 or a derivative thereof (e.g., A83-01). In certain embodiments, the TGFβ/Activin-Nodal inhibitor comprises SB431542.

In certain embodiments, the cells are contacted with or exposed to a BMP inhibitor. In certain embodiments, the concentration of the BMP inhibitor contacted with or exposed to the cells is between about 50 nM and about 500 nM, or between about 100 nM and about 500 nM, or between about 200 nM and about 500 nM, or between about 200 and about 300 nM, or between about 200 nM and about 400 nM, or between about 100 nM and about 250 nM, or between about 100 nM and about 250 nM, or between about 200 nM and about 250 nM, or between about 250 nM and about 300 nM. In certain embodiments, the concentration of the BMP inhibitor contacted with or exposed to the cells is between about 200 nM and about 300 mM. In certain embodiments, the concentration of the BMP inhibitor contacted with or exposed to the cells is about 150 nM, about 200 nM, about 250 nM, about 300 nM, or about 350 nM. In certain embodiments, the concentration of the BMP inhibitor contacted with or exposed to the cells is about 250 nM. In certain embodiments, the BMP inhibitor comprises LDN193189 or a derivative thereof. In certain embodiments, the BMP inhibitor comprises LDN193189.

In certain embodiments, the cells are contacted with or exposed to the TGFβ/Activin-Nodal inhibitor and the BMP inhibitor simultaneously. In certain embodiments, the stem cells are contacted with or exposed to the TGFβ/Activin-Nodal inhibitor and the BMP inhibitor for 7 days. In certain embodiments, the cells are contacted with or exposed to the TGFβ/Activin-Nodal inhibitor and the BMP inhibitor from day 0 through day 6. In certain embodiments, the TGFβ/Activin-Nodal inhibitor and the BMP inhibitor are added every day or every other day to a cell culture medium comprising the stem cells from day 0 through day 6. In certain embodiments, the TGFβ/Activin-Nodal inhibitor and the BMP inhibitor are added every day (daily) to a cell culture medium comprising the stem cells from day 0 to day 6.

### 5.2.3. Wnt Activators

In certain embodiments, the at least one Wnt activator lowers GSK3β for activation of Wnt signaling. Thus, in certain embodiments, the Wnt activator is a GSK3β inhibitor. A GSK3P inhibitor is capable of activating a WNT signaling pathway, see e.g., Cadigan, et al., J Cell Sci. 2006; 119:395-402; Kikuchi, et al., Cell Signaling. 2007; 19:659-671, which are incorporated by reference herein in their entireties. As used herein, the term “glycogen synthase kinase 33 inhibitor” or “GSK3β inhibitor” refers to a compound that inhibits a glycogen synthase kinase 33 enzyme, for example, see Doble, et al., J Cell Sci. 2003; 116:1175-1186, which is incorporated by reference herein in its entirety.

Non-limiting examples of Wnt activators or GSK3β inhibitors include CHIR99021, Wnt3A, Wnt1, Wnt5a, BIO ((3E)-6-bromo-3-[3-(hydroxyamino)indol-2-ylidene]-1H-indol-2-one), CHIR98014, Lithium, 3F8, and those disclosed in WO2011/149762, WO13/067362, Chambers et al., Nat Biotechnol. 2012 Jul. 1; 30(7):715-20, Kriks et al., Nature. 2011 Nov. 6; 480(7378):547-51, and Calder et al., J Neurosci. 2015 Aug. 19; 35(33):11462-81, all of which are incorporated by reference in their entireties. In certain embodiments, the at least one Wnt activator is a small molecule selected from CHIR99021, Wnt3A, Wnt1, Wnt5a, BIO, CHIR98014, Lithium, 3F8, derivatives thereof, and mixtures thereof. In certain embodiments, the at least one Wnt activator comprises CHIR99021 or a derivative thereof.

In certain embodiments, the at least one Wnt activator comprises CHIR99021. “CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl)pyrimidin-2-ylamino) ethylamino)nicotinonitrile with the following formula.

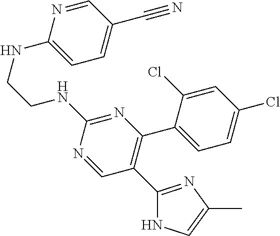 [SMILES: Cc1c[nH]c(-c2cnc(NCCNc3ccc(C#N)cn3)nc2-c2ccc(Cl)cc2Cl)n1]

CHIR99021 is highly selective, showing nearly thousand-fold selectivity against a panel of related and unrelated kinases, with an IC50=6.7 nM against human GSK3β and nanomolar IC50 values against rodent GSK3β homologs.

In certain embodiments, the cells are contacted with or exposed to the at least one Wnt activator for at least about 5 days, at least about 10 days, at least about 15 days, or at least about 20 days. In certain embodiments, the cells are contacted with or exposed to the at least one Wnt activator for up to about 5 days, up to about 10 days, up to about 15 days, or up to about 20 days. In certain embodiments, the cells are contacted with or exposed to the at least one Wnt activator for between about 5 days and about 20 days, between about 5 days and about 15 days, between about 10 days and about 20 days, between about 5 days and about 15 days, or between about 10 days and about 15 days. In certain embodiments, the cells are contacted with the at least one Wnt activator for between about 10 days and about 15 days. In certain embodiments, the cells are contacted with the at least one Wnt activator for about 10 days. In certain embodiments, the stem cells are contacted with the at least one activator of Wnt signaling for 12 days. In certain embodiments, the cells are contacted with the at least one Wnt activator from day 0 through day 11. In certain embodiments, the at least one Wnt activator is added every day or every other day to a cell culture medium comprising the cells from day 0 through day 11. In certain embodiments, the at least one Wnt activator is added every day (daily) to a cell culture medium comprising the cells from day 0 through day 11.

In certain embodiments, the concentration of the at least Wnt activator is increased during its exposure to the cells (also referred to as “Wnt Boost”). In certain embodiments, the increase or Wnt Boost is initiated at least about 2 days, at least about 4 days, or at least about 5 days from the initial exposure of the cells to the at least one Wnt activator. In certain embodiments, the increase or Wnt Boost is initiated about 4 days from the initial exposure of the cells to the at least one Wnt activator.

In certain embodiments, the cells are contacted with or exposed to the increased concentration of the at least one Wnt activator for at least about 5 days, or at least about 10 days. In certain embodiments, the cells are contacted with or exposed to the increased concentration of the at least one Wnt activator for at least about 5 days. In certain embodiments, the cells are contacted with the increased concentration of the at least one Wnt activator for up to about 5 days, up to about 10 days, or up to about 15 days. In certain embodiments, the cells are contacted with the increased concentration of the at least one Wnt activator for up to about 10 days.

In certain embodiments, the cells are contacted with or exposed to the increased concentration of the at least one Wnt activator for between about 5 days and about 15 days, or between about 5 days and about 10 days, or between about 10 days and about 15 days. In certain embodiments, the cells are contacted with or exposed to the increased concentration of the at least one Wnt activator for between about 5 days and about 10 days. In certain embodiments, the cells are contacted with or exposed to the increased concentration of the at least one Wnt activator for about 5 days, about 10 days, or about 15 days. In certain embodiments, the cells are contacted with or exposed to the increased concentration of the at least one Wnt activator for about 5 days. In certain embodiments, the cells are contacted with or exposed to the increased concentration of the at least one Wnt activator for 6 days. In certain embodiments, the cells are contacted with or exposed to the increased concentration of the at least one Wnt activator from day 4 through day 9. In certain embodiments, the cells are contacted with or exposed to the increased concentration of the at least one Wnt activator for about 10 days. In certain embodiments, the cells are contacted with or exposed to the increased concentration of the at least one Wnt activator for 8 days. In certain embodiments, the cells are contacted with or exposed to the increased concentration of the at least one Wnt activator from day 4 through day 11.

In certain embodiments, the initial concentration of the at least one Wnt activator contacted with or exposed to the cells prior to the Wnt Boost is less than about 5 μM, less than about 3 μM, or less than about 1 μM, including, but not limited to, between about 0.01 μM and about 5 μM, between about 0.01 μM and about 3 μM, between about 0.05 μM and about 3 μM, between about 0.1 μM and about 3 μM, between about 0.5 μM and about 3 μM, between about 0.5 μM and about 2 μM, or between about 0.5 μM and about 1 μM. In certain embodiments, the initial concentration of the at least one Wnt activator contacted with or exposed to the cells prior to the Wnt Boost is less than about 1 μM, e.g., about 0.1 μM, about 0.2 μM, about 0.3 μM, about 0.4 μM, about 0.5 μM, about 0.6 μM, about 0.7 μM, about 0.8 μM, about 0.9 μM, or about 1 μM. In certain embodiments, the initial concentration of the at least one Wnt activator contacted with or exposed to the cells prior to the Wnt boost is about 0.5 μM. In certain embodiments, the initial concentration of the at least one Wnt activator contacted with or exposed to the cells prior to the Wnt boost is about 0.7 μM.

In certain embodiments, the increased concentration of the at least one Wnt activator post the Wnt Boost is about 3 μM or greater, about 5 μM or greater, about 10 μM or greater, about 15 μM or greater, or about 20 μM or greater. In certain embodiments, the increased concentration of the at least one Wnt activator post the Wnt Boost is between about 3 μM and about 15 μM, between about 3 μM and about 10 μM, or between about 5 μM and about 10 μM. In certain embodiments, the increased concentration of the at least one Wnt activator post the Wnt Boost is about 3 μM, about 3.5 μM, about 4 μM, about 4.5 μM, about 5 μM, about 5.5 μM, about 6 μM, about 6.5 μM, about 7 μM, about 7.5 μM, about 8 μM, about 8.5 μM, about 9 μM, about 9.5 μM, or about 10 μM. In certain embodiments, the increased concentration of the at least one Wnt activator post the Wnt Boost is about 3 μM. In certain embodiments, the increased concentration of the at least one Wnt activator post the Wnt boost is about 7 μM. In certain embodiments, the increased concentration of the at least one Wnt activator post the Wnt Boost is about 7.5 μM.

In certain embodiments, the concentration of the at least one Wnt activator is increased from the initial concentration contacted with or exposed to the cells by between about 50% and about 2000%, or between about 100% and about 1500%, or between about 150% and about 1500%, or between about 200% and about 1500%, or between about 250% and about 1500%, or between about 300% and about 1500%, or between about 300% and about 1000%, or between about 300% and about 400%, or between about 500% and about 1000%, or between about 800% and about 1000%, or between about 900% and about 1000%, or between about 950% and about 1000. In certain embodiments, the concentration of the at least one Wnt activator is increased from the initial concentration contacted with or exposed to the cells by between about 300% and about 1000%. In certain embodiments, the concentration of the at least one Wnt activator is increased from the initial concentration contacted with or exposed to the cells by between about 300% and about 400%. In certain embodiments, the concentration of the at least one Wnt activator is increased from the initial concentration contacted with or exposed to the cells by between about 900% and about 1000%. In certain embodiments, the concentration of the at least one Wnt activator is increased from the initial concentration contacted with or exposed to the cells by about 300%, about 350%, about 400%, about 450%, about 500%, about 550%, about 600%. 650%, about 700%, about 750%, about 800%, about 850%, about 900%, about 950%, about 1000%, about 1050%, or about 1100%. In certain embodiments, the concentration of the at least one Wnt activator is increased from the initial concentration contacted with or exposed to the cells by about 300%. In certain embodiments, the concentration of the at least one Wnt activator is increased from the initial concentration contacted with or exposed to the cells by about 350%. In certain embodiments, the concentration of the at least one Wnt activator is increased from the initial concentration contacted with or exposed to the cells by about 950%. In certain embodiments, the concentration of the at least one Wnt activator is increased from the initial concentration contacted with or exposed to the cells by about 1000%.

In certain embodiments, the at least one Wnt activator comprises a GSK3β inhibitor. In certain embodiments, the at least one Wnt activator comprises CHIR99021 or a derivative thereof. In certain embodiments, the at least one Wnt activator comprises CHIR99021.

### 5.2.4. SHH Activators

As used herein, the term “Sonic hedgehog,” “SHH,” or “Shh” refers to a protein that is one of at least three proteins in the mammalian signaling pathway family called hedgehog, another is desert hedgehog (DHH) wile a third is Indian hedgehog (IHH). SHH interacts with at least two transmembrane proteins by interacting with transmembrane molecules Patched (PTC) and Smoothened (SMO). SHH typically binds to PTC, which then allows the activation of SMO as a signal transducer. In the absence of SHH, PTC typically inhibits SMO, which in turn activates a transcriptional repressor so transcription of certain genes does not occur. When SHH is present and binds to PTC, PTC cannot interfere with the functioning of SMO. With SMO uninhibited, certain proteins are able to enter the nucleus and act as transcription factors allowing certain genes to be activated (see Gilbert, 2000 Developmental Biology (Sunderland, Mass., Sinauer Associates, Inc., Publishers). In certain embodiments, an SHH activator refers to any molecule or compound that is capable of activating a SHH signaling pathway, including a molecule or compound that is capable of binding to PTC or a SMO. In certain embodiments, the SHH activator is selected from molecules that bind to PCT, molecules that bind to SMO, and combinations thereof. Non-limiting examples of SHH activators include those disclosed in WO10/096496, WO13/067362, Chambers et al., Nat Biotechnol. 2009 March; 27(3):275-80, and Kriks et al., Nature. 2011 Nov. 6; 480(7378):547-51. In certain embodiments, the SHH activator comprises a SHH protein, a SMO agonist, or a combination thereof. In certain embodiments, the SHH protein comprises a recombinant SHH, a purified SHH, or a combination thereof. In certain embodiments, the recombinant SHH comprises a recombinant protein that is at least about 80%, about 85%, about 90%, about 95%, or about 99% identical to a mouse SHH N-terminal fragment. In certain embodiments, the recombinant SHH comprises SHH C25II. In certain embodiments, the SMO agonist comprises purmorphamine.

In certain embodiments, the cells are contacted with or exposed to the at least one SHH activator for at least about 5 days, or at least about 10 days. In certain embodiments, the cells are contacted with or exposed to the at least one SHH activator for up to about 5 days, or up to about 10 days. In certain embodiments, the cells are contacted with or exposed to the at least one SHH activator for between about 5 days and about 10 days. In certain embodiments, the cells are contacted with or exposed to the at least one SHH activator for about 5 days. In certain embodiments, the cells are contacted with or exposed to the at least one SHH activator for 7 days. In certain embodiments, the cells are contacted with or exposed to the at least one SHH activator from day 0 through day 6. In certain embodiments, the at least one SHH activator is added every day or every other day to a cell culture medium comprising the cells from day 0 through day 6. In certain embodiments, the at least one SHH activator is added every day (daily) to a cell culture medium comprising the cells from day 0 through day 6.

In certain embodiments, the concentration of the at least one SHH activator contacted with or exposed to the cells is between about 50 ng/mL and about 1000 ng/mL, between about 100 ng/mL and about 1000 ng/mL, between about 20 ng/mL and about 1000 ng/mL, between about 300 ng/mL and about 1000 ng/mL, between about 400 ng/mL and about 1000 ng/mL, between about 500 ng/mL and about 1000 ng/mL, between about 400 ng/mL and about 800 ng/mL, between about 400 ng/mL and about 700 ng/mL, between about 400 ng/mL and about 600 ng/mL, or between about 500 ng/mL and about 600 ng/mL. In certain embodiments, the concentration of the at least one SHH activator contacted with or exposed to the cells is between about 400 ng/mL and about 600 ng/mL. In certain embodiments, the concentration of the at least one SHH activator contacted with or exposed to the cells is about 400 ng/mL, about 450 ng/mL, about 500 ng/mL, about 550 ng/mL, or about 600 ng/mL. In certain embodiments, the concentration of the at least one SHH activator contacted with or exposed to the cells is about 500 ng/mL.

In certain embodiments, the at least one activator of SHH signaling comprises SHH C25II.

### 5.2.5. FGF Activators

FGF family includes secreted signaling proteins (secreted FGFs) that signal to receptor tyrosine kinases. Phylogenetic analysis suggests that 22 Fgfgenes can be arranged into seven subfamilies containing two to four members each. Branch lengths are proportional to the evolutionary distance between each gene.

In certain embodiments, the FGF activator is selected from FGF8a, FGF17, FGF18, FGF8b, FGF2, FGF4, and derivatives thereof. In certain embodiments, the FGF activator is selected from FGF8a, FGF17, FGF18, FGF2, FGF4, and derivatives thereof. In certain embodiments, the FGF activator is selected from FGF8a, FGF17, FGF18.

The FGF8 subfamily is comprised of FGF8a, FGF8b, FGF17, and FGF18. Early patterning of the vertebrate midbrain and cerebellum is regulated by a mid/hindbrain organizer that produces FGF8a, FGF8b, FGF17 and FGF18. It has been shown that FGF8b functions differently from FGF8a, FGF17, and FGF18 (Liu et al., Development. 2003 December; 130(25):6175-85). FGF8b is the only protein that can induce the r1 gene Gbx2 and strongly activate the pathway inhibitors Spry1/2, as well as repress the midbrain gene Otx2 (Liu 2003). Moreover, FGF8b extends the organizer along the junction between the induced Gbx2 domain and the remaining Otx2 region in the midbrain, correlating with cerebellum development (Liu 2003). By contrast, FGF8a, FGF17, and FGF18 cause expansion of the midbrain and upregulating midbrain gene expression (Liu 2003).

In certain embodiments, the FGF activator is capable of causing expansion of the midbrain and upregulating midbrain gene expression. In certain embodiments, the FGF activator is selected from FGF8a, FGF17, FGF18, FGF2, FGF4, derivatives thereof, and combinations thereof. In certain embodiments, the FGF activator comprises or is FGF18.

In certain embodiments, the cells are contacted with or exposed to the at least one FGF activator for at least about 1 day, at least about 3 days, at least about 5 days, at least about 8 days, or at least about 10 days. In certain embodiments, the cells are contacted with or exposed to the at least one FGF activator for up to about 5 days, or up to about 10 days, or up to about 15 days, or up to about 20 days. In certain embodiments, the cells are contacted with or exposed to the at least one FGF activator for between about 1 days and about 20 days, between about 1 day and about 15 days, or between about 5 days and about 20 days, or between about 5 days and about 15 days, or between about 5 days and about 10 days, or between about 10 days and about 20 days. In certain embodiments, the cells are contacted with or exposed to the at least one FGF activator for between about 5 days and about 10 days. In certain embodiments, the cells are contacted with or exposed to the at least one FGF activator for about 3 days, about 5 days, or about 8 days. In certain embodiments, the cells are contacted with or exposed to the at least one FGF activator for about 5 days.

In certain embodiments, the initial contact of the cells with or the initial exposure of the cells to the at least one FGF activator is at least about 5 days, or at least about 10 days from the initial contact of the cells with or the initial exposure of the cells to the at least one SMAD inhibitor. In certain embodiments, the initial contact of the cells with or the initial exposure of the cells to the at least one FGF activator is no later than about 5 days, no later than about 10 days, or no later than about 15 days from the initial contact of the cells with or the initial exposure of the cells to the at least one SMAD inhibitor. In certain embodiments, the initial contact of the cells with or the initial exposure of the cells to the at least one FGF activator is between about 5 days and about 15 days, between about 5 days and about 10 days, or between about 10 days and about 15 days, from the initial contact of the cells with or the initial exposure of the cells to the at least one SMAD inhibitor. In certain embodiments, the initial contact of the cells with or the initial exposure of the cells to the at least one FGF activator is between about 5 days and about 10 days from the initial contact of the cells with or the initial exposure of the cells to the at least one SMAD inhibitor. In certain embodiments, the initial contact of the cells with or the initial exposure of the cells to the at least one FGF activator is about 10 days from the initial contact of the cells with or the initial exposure of the cells to the at least one SMAD inhibitor. In certain embodiments, the initial contact of the cells with or the initial exposure of the cells to the at least one FGF activator is 9 days from the initial contact of the cells with or the initial exposure of the cells to the at least one SMAD inhibitor. In certain embodiments, the initial contact of the cells with or the initial exposure of the cells to the at least one FGF activator is 10 days from the initial contact of the cells with or the initial exposure of the cells to the at least one SMAD inhibitor. In certain embodiments, the initial contact of the cells with or the initial exposure of the cells to the at least one FGF activator is 12 days from the initial contact of the cells with or the initial exposure of the cells to the at least one SMAD inhibitor.

In certain embodiments, the initial contact of the cells with or the initial exposure of the cells to the at least one FGF activator is about 5 days from the initial contact of the cells with or the initial exposure of the cells to the at least one SMAD inhibitor, and the cells are contacted with the at least FGF activator for about 3 days. In certain embodiments, the initial contact of the cells with or the initial exposure of the cells to the at least one FGF activator is about 5 days from the initial contact of the cells with or the initial exposure of the cells to the at least one SMAD inhibitor, and the cells are contacted with the at least FGF activator for about 5 days. In certain embodiments, the initial contact of the cells with or the initial exposure of the cells to the at least one FGF activator is about 10 days from the initial contact of the cells with or the initial exposure of the cells to the at least one SMAD inhibitor, and the cells are contacted with the at least FGF activator for about 3 days. In certain embodiments, the initial contact of the cells with or the initial exposure of the cells to the at least one FGF activator is about 10 days from the initial contact of the cells with or the initial exposure of the cells to the at least one SMAD inhibitor, and the cells are contacted with the at least FGF activator for about 5 days. In certain embodiments, the initial contact of the cells with or the initial exposure of the cells to the at least one FGF activator is 12 days from the initial contact of the cells with or the initial exposure of the cells to the at least one SMAD inhibitor, and the cells are contacted with the at least FGF activator for 5 days.

In certain embodiments, the concentration of the at least one FGF activator contacted with or exposed to the cells is between about 10 ng/mL and about 500 ng/mL, between about 50 ng/mL and about 500 ng/mL, between about 100 ng/mL and about 500 ng/mL, between about 100 ng/mL and about 400 ng/mL, between about 100 ng/mL and about 300 ng/mL, between about 100 ng/mL and about 200 ng/mL, between about 100 ng/mL and about 250 ng/mL. In certain embodiments, the concentration of the at least one FGF activator contacted with or exposed to the cells is between about 100 ng/mL and about 200 ng/mL In certain embodiments, the concentration of the at least one FGF activator contacted with or exposed to the cells is about 100 ng/mL. In certain embodiments, concentration of the at least one FGF activator contacted with or exposed to the cells is about 200 ng/mL.

In certain embodiments, the at least one FGF activator comprises FGF18.

In certain non-limiting embodiments, the stem cells are contacted with or exposed to at least one TGFβ/Activin-Nodal inhibitor (e.g., SB431542, e.g., at a concentration of about 10 μM), at least one BMP inhibitor (e.g., LDN193189, e.g., at a concentration of about 250 nM), and at least one SHH activator (e.g., SHH C25II, e.g., a concentration of about 500 ng/mL) for about 5 days (e.g., 7 days, e.g., from day 0 to day 6), and the cells are contacted with the at least one Wnt activator (e.g., CHIR99021, e.g., at a concentration of about 0.7 μM for 5 days (e.g., 4 days, e.g., from day 0 to day 3), and at a concentration of about 7.5 μM for about 5 days (e.g., 6 days, e.g., from day 4 to day 9), and at a concentration of about 3 μM for about 2 days (e.g., from day 10 to day 11). The cells are contacted with or exposed to the at least one FGF activator (e.g., FGF18, e.g., at a concentration of about 100 ng/ml), wherein the initial contact of the cells with the at least one FGF activator is about 10 days (e.g., 10 days or 12 days) from the initial contact of the cells with the at least one SMAD inhibitor, and the cells are contacted with the at least one FGF activator for about 5 days (e.g., e.g., 5 days (from day 12 to day 16) or 7 days (e.g., from day 10 to day 16).

### 5.2.6. Cell Culture Media

In certain embodiments, the above-described inhibitors and activators are added to a cell culture medium comprising the cells. Suitable cell culture media include, but are not limited to, Knockout® Serum Replacement (“KSR”) medium, Neurobasal® medium (NB), N2 medium, B-27 medium, and Essential 8®/Essential 6® (“E8/E6”) medium, and combinations thereof. KSR medium, NB medium, N2 medium, B-27 medium, and E8/E6 medium are commercially available. KSR medium is a defined, serum-free formulation optimized to grow and maintain undifferentiated hESCs in culture.

In certain embodiments, the cell culture medium is a KSR medium. The components of a KSR medium are disclosed in WO2011/149762. In certain embodiments, a KSR medium comprises Knockout DMEM, Knockout Serum Replacement, L-Glutamine, Pen/Strep, MEM, and 13-mercaptoethanol. In certain embodiments, 1 liter of KSR medium comprises 820 mL of Knockout DMEM, 150 mL of Knockout Serum Replacement, 10 mL of 200 mM L-Glutamine, 10 mL of Pen/Strep, 10 mL of 10 mM MEM, and 55 μM of 13-mercaptoethanol.

In certain embodiments, the cell culture medium is an E8/E6 medium. E8/E6 medium is a feeder-free and xeno-free medium that supports the growth and expansion of human pluripotent stem cells. E8/E6 medium has been proven to support somatic cell reprogramming. In addition, E8/E6 medium can be used as a base for the formulation of custom media for the culture of PSCs. One example E8/E6 medium is described in Chen et al., Nat Methods 2011 May; 8(5):424-9, which is incorporated by reference in its entirety. One example E8/E6 medium is disclosed in WO15/077648, which is incorporated by reference in its entirety. In certain embodiments, an E8/E6 cell culture medium comprises DMEM/F12, ascorbic acid, selenium, insulin, NaHCO₃, transferrin, FGF2 and TGFβ. The E8/E6 medium differs from a KSR medium in that E8/E6 medium does not include an active BMP or Wnt ingredient. Thus, in certain embodiments, when an E8/E6 medium is used to culture the presently disclosed population of stem cells to differentiate into a population of proprioceptors, at least one inhibitor of SMAD signaling (e.g., those inhibiting BMP) is not required to be added to the E8/E6 medium.

### 5.2.7. Differentiated Cells

In certain embodiments, the method comprises obtaining a cell population of the differenced cells, wherein at least about 10%, at least about 20%, at least about 30%, at least about 40%, at least about 50%, at least about 60%, at least about 70%, at least about 80%, or at least about 90% of the differentiated cells express at least one marker indicating a mDA or a mDA precursor. Non-limiting examples of markers indicating a mDA or a mDA precursor include engrailed-1 (EN1), orthodenticle homeobox 2 (OTX2), tyrosine hydroxylase (TH), nuclear receptor related-1 protein (NURR1), forkhead box protein A2 (FOXA2), and LIM homeobox transcription factor 1 alpha (LMX1A), PITX3, LMO3, SNCA, ADCAP1, CHRNA4, and GIRK2.

In certain embodiments, the differentiated cells express the at least one marker indicating a mDA or a mDA precursor at least about 10 days (e.g., about 15 days (e.g., 16 days), about 20 days, about 30 days, about 40 days, or about 50 days) from the initial contact of the cells with the at least one SMAD inhibitor.

The treatment of the cells with at least FGF activator can lead to sustained expression of EN1. EN1 is a survival factor for midbrain DA neurons during development, and continues to exert neuroprotective and physiological function in adult midbrain DA neurons. As such, cells with sustained expression of EN1 can develop into functionally mature mDA upon further development and maturation. In certain embodiments, the differentiated cells have a detectable level of expression of EN1 at least about 10 days, at least about 15 days, at least about 16 days, at least about 20 days, at least about 25 days, at least about 27 days, at least about 30 days, at least about 35 days, at least about 40 days, at least about 45 days, at least about 50 days, at least about 60 days, at least about 70 days, at least about 80 days, or at least about 90 days from the initial contact of the stem cells to the at least one SMAD inhibitor. In certain embodiments, the differentiated cells have a detectable level of expression of EN1 about 30 days from the initial contact of the stem cells to the at least one SMAD inhibitor. In certain embodiments, the differentiated cells have a detectable level of expression of EN1 about 40 days from the initial contact of the stem cells to the at least one SMAD inhibitor.

In certain embodiments, the differentiated cells derived from the presently disclosed methods do not express or have a low expression of at least one marker selected from PAX6, EMX2, LHX2, SMA, SIX1, PITX2, SIM1, POU4F1, PHOX2A, BARHL1, BARHL2, GBX2, HOXA2, HOXB2, POUSF1, NANOG, and combinations thereof.

In certain embodiments, the cells are contacted with the activators and inhibitors described herein at a concentration and for a time effective to increase a detectable level of expression of at least one of marker of a DA neuron, for example, EN1, or wherein the cells are A9 type neuronal cells.

In certain embodiments, the cells are contacted with the activators and inhibitors described herein at a concentration and time effective to decrease expression of SMA, SIX1, PITX2, SIM1, POU4F1, and/or PHOX2A.

5.2.8. Differentiation of mDA Precursors to mDAs

In certain embodiments, the cells are further contacted with DA neuron lineage specific activators and inhibitors, for example, L-glutamine, brain-derived neurotrophic factor (BDNF), glial cell-derived neurotrophic factor (GDNF), Cyclic adenosine monophosphate (cAMP), Transforming growth factor beta (TGFβ, for example, TGFβ3), ascorbic acid (AA), and DAPT (which is also known as, N-[(3,5-Difluorophenyl)acetyl]-L-alanyl-2-phenyl]glycine-1,1-dimethylethyl ester; LY-374973, N—[N-(3,5-Difluorophenacetyl)-L-alanyl]-S-phenylglycine t-butyl ester; or N—[N-(3,5-difluorophenacetyl)-L-alanyl]-S-phenylglycine t-butyl ester). In certain embodiments, the cells are contacted with the foregoing DA neuron lineage specific activators and inhibitors for at least about 2, at least about 3, at least about 4, at least about 5, at least about 6, at least about 7, at least about 8, at least about 9, or at least about 10 or more days, for example, between about 2 days and about 20 days, between about 3 days and about 19 days, between about 4 days and about 18 days, between about 5 days and about 17 days, between about 6 days and about 16 days, between about 7 days and about 15 days, between about 8 days and about 15 days, between about 9 days and about 14 days, or between about 10 days and about 13 days. In certain embodiments, the cells are contacted with the foregoing DA neuron lineage specific activators and inhibitors for up to about 2, up to about 3, up to about 4, up to about 5, up to about 6, up to about 7, up to about 8, up to about 9, or up to about 10 days or more days. In certain embodiments, the cells are contacted with the foregoing DA neuron lineage specific activators and inhibitors for about 4 days, about 5 days, about 6 days, about 7 days, or about 8 days.

In certain embodiments, the cells are contacted with L-glutamine at a concentration of between about 0.5 mM and about 5 mM, or between about 1 mM and about 5 mM, or between about 1.5 mM and about 2.5 mM, or between about 1 mM and about 2 mM. In certain embodiments, the cells are contacted with L-glutamine at a concentration of about 2 mM.

In certain embodiments, the cells are contacted with BDNF at a concentration of between about 5 ng/ml and about 50 ng/mL, or between about 10 ng/ml and about 50 ng/mL, or between about 10 ng/ml and about 40 ng/mL, or between about 20 ng/ml and about 50 ng/mL, or between about 20 ng/ml and about 40 ng/mL, or between about 10 ng/ml and about 30 ng/mL, or between about 10 ng/ml and about 20 ng/mL, or between about 20 ng/ml and about 30 ng/mL. In certain embodiments, the cells are contacted with BDNF at a concentration of about 20 ng/mL.

In certain embodiments, the cells are contacted with ascorbic acid (AA) at a concentration of between about 50 nM and about 500 nM, or between about 100 nM and about 500 nM, or between about 100 nM and about 400 nM, or between about 200 nM and about 400 nM, or between about 200 nM and about 300 nM, or between about 100 nM and about 300 nM. In certain embodiments, the cells are contacted with AA at a concentration of about 200 nM.

In certain embodiments, the cells are contacted with GDNF at a concentration of between about 5 ng/ml and about 50 ng/mL, or between about 10 ng/ml and about 50 ng/mL, or between about 10 ng/ml and about 40 ng/mL, or between about 20 ng/ml and about 50 ng/mL, or between about 20 ng/ml and about 40 ng/mL, or between about 10 ng/ml and about 30 ng/mL, or between about 10 ng/ml and about 20 ng/mL, or between about 20 ng/ml and about 30 ng/mL. In certain embodiments, the cells are contacted with GDNF at a concentration of about 20 ng/mL.

In certain embodiments, the cells are contacted with cAMP at a concentration of between about 200 nM and about 800 nM, or between about 200 nM and about 700 nM, or between about 300 nM and about 700 nM, or between about 300 nM and about 600 nM, or between about 400 nM and about 600 nM, or between about 450 nM and about 550 nM. In certain embodiments, the cells are contacted with cAMP at a concentration of about 500 nM.

In certain embodiments, the cells are contacted with TGFβ3 at a concentration of between about 0.01 ng/ml and about 5 ng/mL, or between about 0.1 ng/ml and about 4 ng/mL, or between about 0.5 ng/ml and about 5 ng/mL, or between about 1 ng/ml and about 3 ng/mL, or between about 1 ng/ml and about 2 ng/mL. In certain embodiments, the cells are contacted with TGFβ3 at a concentration of about 1 ng/mL.

In certain embodiments, the differentiated midbrain DA precursors are further cultured as described by U.S. Publication No. 2015/0010514, which is incorporated by reference in its entirety.

### 5.3. Methods of Isolating Midbrain DA Neurons and Precursor Thereof

The present disclosure provides methods for isolating mDAs and precursors thereof based on at least one or at least two surface markers. In certain embodiments, the surface marker is a negative surface marker, wherein the cells do not express a detectable level of the negative surface marker. In certain embodiments, the cells express a reduced level of the negative surface marker as compared to the mean expression level of the negative surface marker of a population of cells of which the cells are isolated from.

In certain embodiments, the surface marker is a positive surface marker, wherein the cells express a detectable level of the positive surface marker. In certain embodiments, the cells express an increased level of the positive surface marker as compared to the mean expression level of the positive surface marker of a population of cells of which the cells are isolated from.

In certain embodiments, the presently disclosed method for isolating mDAs and precursors thereof from a population of cells comprises isolating cells that do not express a detectable level of at least one negative surface marker. In certain embodiments, the presently disclosed method for isolating mDAs and precursors thereof from a population of cells comprises isolating cells that do not express a detectable level or express a reduced level of at least one negative surface marker as compared to the mean expression level of the at least one negative surface marker in the population of cells. In certain embodiments, the presently disclosed method for isolating mDAs and precursors thereof from a population of cells comprises isolating cells that express a detectable level of at least one positive surface marker. In certain embodiments, the presently disclosed method for isolating mDAs and precursors thereof from a population of cells comprises isolating cells that express an increased level of at least one positive surface marker as compared to the mean expression level of the at least one positive marker in the population of cells.

In certain embodiments, the presently disclosed method for isolating mDAs and precursors thereof from a population of cells comprises isolating cells that do not express a detectable level of at least one negative surface marker and express a detectable level of at least one positive surface marker. In certain embodiments, the presently disclosed method for isolating mDAs and precursors thereof from a population of cells comprises isolating cells that (a) do not express a detectable level or express a reduced level of at least one negative surface marker as compared to the mean expression level of the at least one negative surface marker in the population of cells; and (b) an increased level of at least one positive surface marker as compared to the mean expression level of the at least one positive marker in the population of cells.

In certain embodiments, the negative surface markers are selected from CD49e (also known as integrin alpha 5), CD99, CD340, and combinations thereof. In certain embodiments, the positive surface markers are selected from CD171, CD184, and combinations thereof.

In certain embodiments, the presently disclosed method for isolating mDAs and precursors thereof from a population of cells comprises isolating cells that do not express a detectable level of CD49e and express a detectable level of CD184. In certain embodiments, the presently disclosed method for isolating mDAs and precursors thereof from a population of cells comprises isolating cells that do not express a detectable level or express a reduced level of CD49e as compared the mean expression level of CD49e in the population of cells; and express an increased level of CD184 as compared to the mean expression level of CD184 in the population of cells.

Any surface-marker based cell isolation technology known in the art can be used in the presently disclosed methods. In certain embodiments, flow cytometry is used to the presently disclosed isolation methods.

### 5.4. Cell Populations and Compositions

The presently disclosure provides a cell population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the cells express at least one marker indicating a mDA or a mDA precursor. Non-limiting examples of markers indicating a mDA or a mDA precursor include EN1, OTX2, TH, NURR1, FOXA2, LMX1A, PITX3, LMO3, SNCA, ADCAP1, CHRNA4, and GIRK2. The presently disclosure also provides compositions comprising such cell populations. In certain embodiments, the in vitro differentiated cells are obtained by the differentiation methods described herewith, for example, in Section 5.2.

In certain embodiments less than about 50% (e.g., less than about 45%, less than about 40%, less than about 35%, less than about 30%, less than about 25%, less than about 20%, less than about 15%, less than about 10%, less than about 5%, less than about 4%, less than about 3%, less than about 2%, less than about 1%, less than about 0.5%, or less than about 0.1%) of the differentiated cells express at least one marker selected from PAX6, EMX2, LHX2, SMA, SIX1, PITX2, SIM1, POU4F1, PHOX2A, BARHL1, BARHL2, GBX2, HOXA2, HOXB2, POUSF1, NANOG, and combinations thereof.

The present disclosure also provides a cell population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the cells express at least one positive surface marker disclosed herein (e.g., in Section 5.3) and do not express at least one negative surface marker disclosed herein (e.g., in Section 5.3). The present disclosure also provides a cell population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the cells express an increased level of at least one positive surface marker disclosed herein (e.g., in Section 5.3) as compared to the mean expression level of the at least one positive marker in the population of cells; and do not express a detectable level or express a reduced level of at least one negative surface marker disclosed herein (e.g., in Section 5.3) as compared to the mean expression level of the at least one negative surface marker in the population of cells.

In certain embodiments, at least about 50% (e.g., at least about 55%, at least about 60%, at least about 70%, at least about 75%, at least about 80%, at least about 85%, at least about 90%, at least about 95%, or at least about 99%) of the cells do not express a detectable level of CD49e and express a detectable level of CD184. In certain embodiments, at least about 50% (e.g., at least about 55%, at least about 60%, at least about 70%, at least about 75%, at least about 80%, at least about 85%, at least about 90%, at least about 95%, or at least about 99%) of the cells do not express a detectable level or express a reduced level of CD49e as compared to the mean expression level of CD49e in the population of cells; and express an increased level of CD184 as compared to the mean expression level of CD184 in the population of cells. In addition, the present disclosure provides compositions comprising such cell populations.

In certain embodiments, the cells are comprised in a composition that further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties). In certain embodiments, the composition further comprises growth factors for promoting maturation of the implanted/grafted cells into midbrain DA cells.

In certain embodiments, the composition comprises a cell population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the cells are administered to a subject. In certain embodiments, from about 1×10⁵ to about 1×10⁷ the cells thereof are administered to a subject.

In certain embodiments, said composition is frozen. In certain embodiments, said composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that comprises a pharmaceutically acceptable carrier. The compositions can be used for preventing and/or treating a neurodegenerative disorders include Parkinson's disease, Huntington's disease, Alzheimer's disease, and multiple sclerosis.

The presently disclosed subject matter also provides a device comprising the differentiated cells or the composition comprising thereof, as disclosed herein. Non-limiting examples of devices include syringes, fine glass tubes, stereotactic needles and cannulas.

### 5.5. Method of Treating Neurodegenerative Disorders

The cell populations and compositions disclosed herein (e.g., those disclosed in Section 5.4) can be used for treating a neurodegenerative disorder. The presently disclosed subject matter provides for methods of treating a neurodegenerative disorder. In certain embodiments, the method comprises administering an effective amount of the presently disclosed stem-cell-derived mDAs or a composition comprising thereof into a subject suffering from a neurodegenerative disorder. In certain embodiments, the method comprises administering an effective amount of the in vitro differentiated cells do not express a detectable level of at least one negative surface marker (e.g., CD49e) and express a detectable level of at least one positive surface marker (e.g., CD184) or a composition comprising such cells into a subject suffering from a neurodegenerative disorder. In certain embodiments, the method comprises administering an effective amount of the in vitro differentiated cells do not express a detectable level or express a reduced level of at least one negative surface marker (e.g., CD49e) as compared to the mean expression of the at least one negative surface marker of a population of cells of which the cells are isolated from, and express an increased level of at least one positive surface marker (e.g., CD184) as compared to the mean expression of the at least one positive surface marker of a population of cells of which the cells are isolated from; or a composition comprising such cells into a subject suffering from a neurodegenerative disorder. In certain embodiments, the composition is a pharmaceutical composition further comprising a pharmaceutically acceptable carrier.

Non-limiting examples of a neurodegenerative disorders include Parkinson's disease, Huntington's disease, Alzheimer's disease, and multiple sclerosis.

In certain embodiments, the neurodegenerative disease is Parkinson's disease. Primary motor signs of Parkinson's disease include, for example, but not limited to, tremor of the hands, arms, legs, jaw and face, bradykinesia or slowness of movement, rigidity or stiffness of the limbs and trunk and postural instability or impaired balance and coordination.

In certain embodiments, the neurodegenerative disease is a parkinsonism disease, which refers to diseases that are linked to an insufficiency of dopamine in the basal ganglia, which is a part of the brain that controls movement. Symptoms include tremor, bradykinesia (extreme slowness of movement), flexed posture, postural instability, and rigidity. Non-limiting examples of parkinsonism diseases include corticobasal degeneration, Lewy body dementia, multiple systematrophy, and progressive supranuclear palsy.

The cells or compositions can be administered or provided systemically or directly to a subject for treating or preventing a neurodegenerative disorder. In certain embodiments, the cells or compositions are directly injected into an organ of interest (e.g., the central nervous system (CNS) or peripheral nervous system (PNS)). In certain embodiments, the cells or compositions are directly injected into the striatum.

The cells or compositions can be administered in any physiologically acceptable vehicle. The cells or compositions can be administered via localized injection, orthotopic (OT) injection, systemic injection, intravenous injection, or parenteral administration. In certain embodiments, the cells or compositions are administered to a subject suffering from a neurodegenerative disorder via orthotopic (OT) injection.

The cells or compositions can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived precursors. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the cells is the quantity of cells necessary to achieve an optimal effect. An optimal effect includes, but is not limited to, repopulation of CNS and/or PNS regions of a subject suffering from a neurodegenerative disorder, and/or improved function of the subject's CNS and/or PNS.

An “effective amount” (or “therapeutically effective amount”) is an amount sufficient to affect a beneficial or desired clinical result upon treatment. An effective amount can be administered to a subject in at least one doses. In terms of treatment, an effective amount is an amount that is sufficient to palliate, ameliorate, stabilize, reverse or slow the progression of the neurodegenerative disorder or pituitary disorder, or otherwise reduce the pathological consequences of the neurodegenerative disorder. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the cells is an amount that is sufficient to repopulate CNS and/or PNS regions of a subject suffering from a neurodegenerative disorder. In certain embodiments, an effective amount of the cells is an amount that is sufficient to improve the function of the CNS and/or PNS of a subject suffering from a neurodegenerative disorder, e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's CNS and/or PNS.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the cells are administered to a subject. In certain embodiments, from about 1×10⁵ to about 1×10⁷ of the cells are administered to a subject suffering from a neurodegenerative disorder. In certain embodiments, from about 1×10⁶ to about 1×10⁷ of the cells are administered to a subject suffering from a neurodegenerative disorder. In certain embodiments, from about 1×10⁶ to about 4×10⁶ of the cells are administered to a subject suffering from a neurodegenerative disorder. The precise determination of what would be considered an effective dose may be based on factors individual to each subject, including their size, age, sex, weight, and condition of the particular subject. Dosages can be readily ascertained by those skilled in the art from this disclosure and the knowledge in the art.

### 5.6. Kits

The presently disclosed subject matter provides kits for inducing differentiation of stem cells to mDAs or precursors thereof. In certain embodiments, the kit comprises (a) at least one inhibitor of SMAD signaling, (b) at least one activator of Wnt signaling, (c) at least one activator of SHH signaling, and (d) at least one activator of FGF signaling. In certain embodiments, the kit further comprises (e) instructions for inducing differentiation of the stem cells into a population of differentiated cells that express at least one marker indicating a mDA or a precursor thereof.

In certain embodiments, the instructions comprise contacting the stem cells with the inhibitor(s), activator(s) and molecule(s) in a specific sequence. The sequence of contacting the inhibitor(s), activator(s) and molecule(s) can be determined by the cell culture medium used for culturing the stem cells.

In certain embodiments, the instructions comprise contacting the stem cells with the inhibitor(s), activator(s) and molecule(s) as described by the methods of the present disclosure (see Section 5.2).

In certain embodiments, the present disclosure provides kits comprising an effective amount of a cell population or a composition disclosed herein in unit dosage form. In certain embodiments, the kit comprises a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit comprises instructions for administering the cell population or composition to a subject suffering from a neurodegenerative disorder. The instructions can comprise information about the use of the cells or composition for treating or preventing a neurodegenerative disorder. In certain embodiments, the instructions comprise at least one of the following: description of the therapeutic agent; dosage schedule and administration for treating or preventing a neurodegenerative disorder or symptoms thereof, precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

### 6. EXAMPLES

The presently disclosed subject matter will be better understood by reference to the following Example, which is provided as exemplary of the presently disclosed subject matter, and not by way of limitation.

### Example 1: Optimization of Midbrain DA Neuron Differentiation Protocol

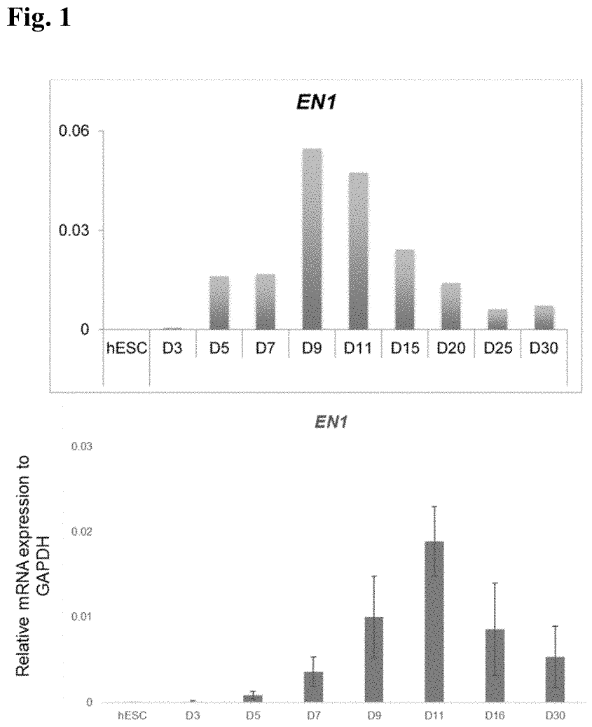

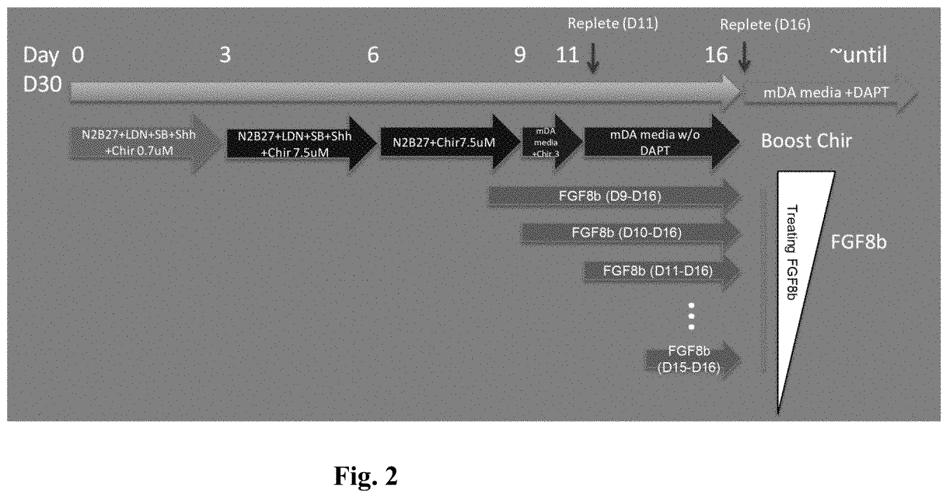

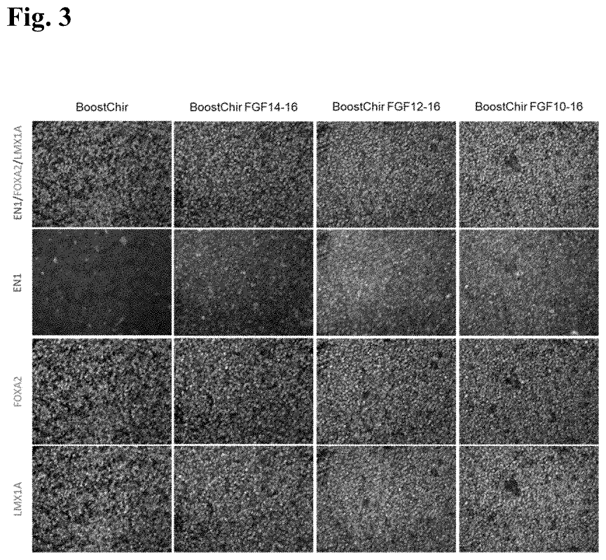

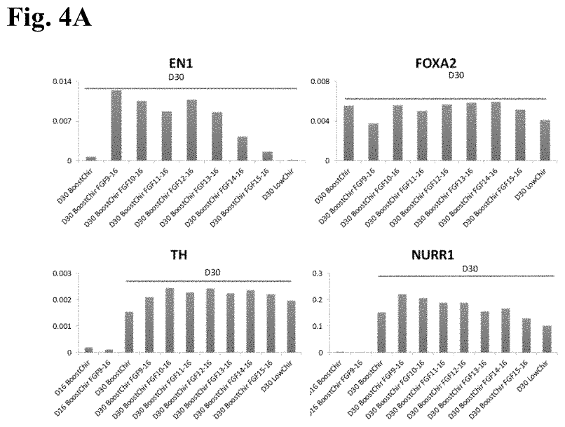

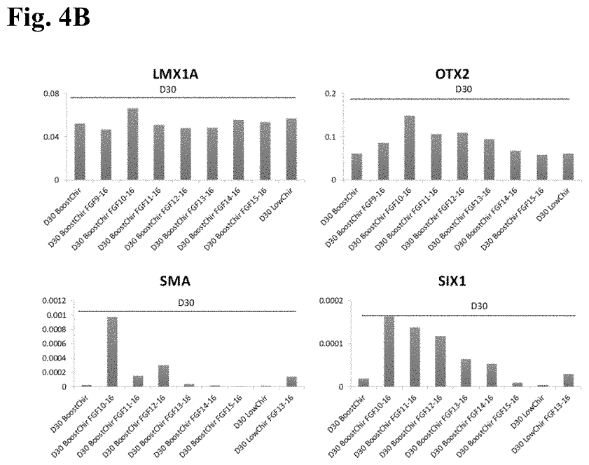

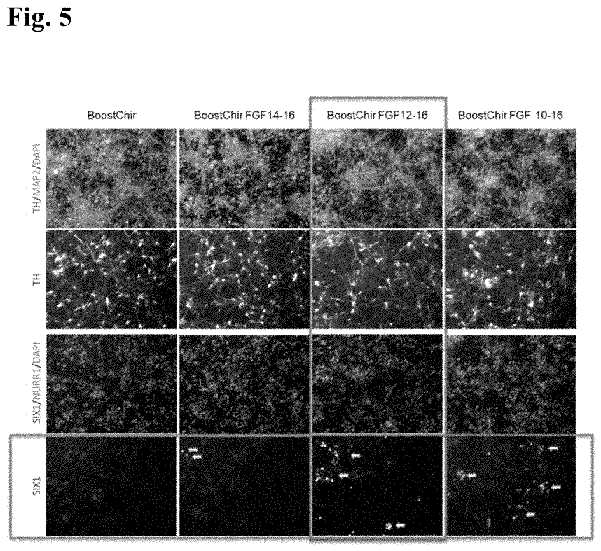

Midbrain DA neurons and precursors thereof were derived from stem cells under a Wnt-Boost protocol previously disclosed (the “7.5 M bump protocol (modification of GMP V2B)” protocol disclosed in International Publication No. WO 2016/196661, which is incorporated by reference in its entirety), which is referred to as “Wnt-Boost protocol” or “Boost protocol” hereinafter. It was discovered that EN1 expression was decreased starting from day 11 of the differentiation with Wnt-Boost protocol (see FIG. 1). Maintaining EN1 expression in the differentiated cells is important to generate mature and functional midbrain DA neurons. Thus, to maintain EN1 expression, the present example tested adding FGF8b treatment to the Wnt-Boost protocol at a late time of differentiation (see FIG. 2). Cells were contacted with FGF8b from day 9 to day 16, from day 10 to day 16, from day 11 to day 16, from day 12 to day 16, from day 13 to day 16, from day 14 to day 16, or from day 15 to day 16, in addition to the Wnt-Boost protocol, were tested. Immunostaining of the cells on day 16 of differentiation shows that EN1 protein expression was maintained in a FGF8b contacting duration dependent manner (see FIG. 3). EN1 positive cells also expressed FOXA2 and LMX1A. RNA expression measured in the cells on day 30 of differentiation shows that the expression levels of mDA or mDA precursor markers FOXA2, NURR1, LMX1A, OXT2, and TH were comparable in all conditions, and the mRNA expression of EN1 was maintained in a FGF8b contacting duration dependent manner (see FIGS. 4A-4B). However, mRNA levels of contamination markers (non-mDA or non-mDA precursor markers), e.g., SMA and SIX1, were also induced in a FGF8b contacting duration dependent manner (see FIG. 4B). SIX1 immuno-staining of the cells on day 30 of differentiation confirmed the mRNA results (see FIG. 5).

FGF8b have been used to derive midbrain DA neurons from pluripotent stem cells. FGF8, FGF17, and FGF18 are subfamily of FGFs, and it has been demonstrated that FGF17b, FGF18 have different role from FGF8b in midbrain development (Liu et al., Development. 2003 December; 130(25):6175-85). It has also been shown that FGF18 can protect again 6-OHDA induced midbrain dopamine neuron damage (Guo et al., Neuroscience. 2017 Jul. 25; 356:229-241). In addition, FGF8b (isthmus and rhombomerel) extends the organizer along the junction between the induced Gbx2 domain and the remaining Otx2 region in the midbrain. FGF8a, FGF17, and FGF18 are responsible for the expansion of the midbrain and up-regulating midbrain gene. FGF8b, FGF17, and FGF18, which are all same FGF subgroup of paracrine FGF to FGF8b.

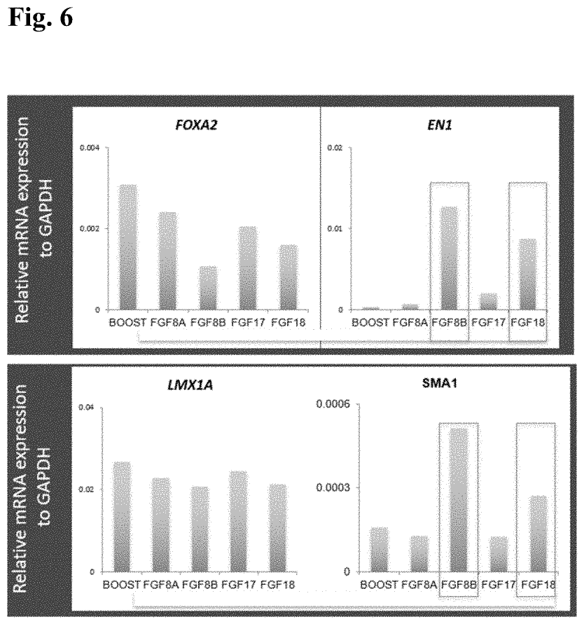

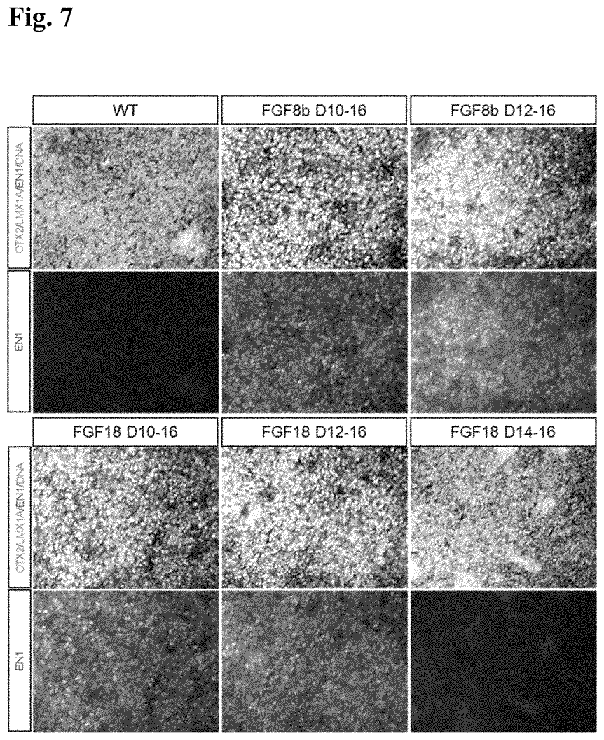

FGF8b, FGF17, and FGF18 were tested by being added them to the cell culture from day 12 to day 16 at a concentration of 100 ng/ml under the WNT-Boost protocol. It was discovered that in the cells on day 16 of differentiation, FGF18 induced the similar mRNA expression level of EN1 as FGF8b, but with reduced mRNA expression level of SMA (see FIGS. 6 and 7). EN1 protein expression was also highly maintained in a FGF18 contacting duration dependent manner as FGF8b.

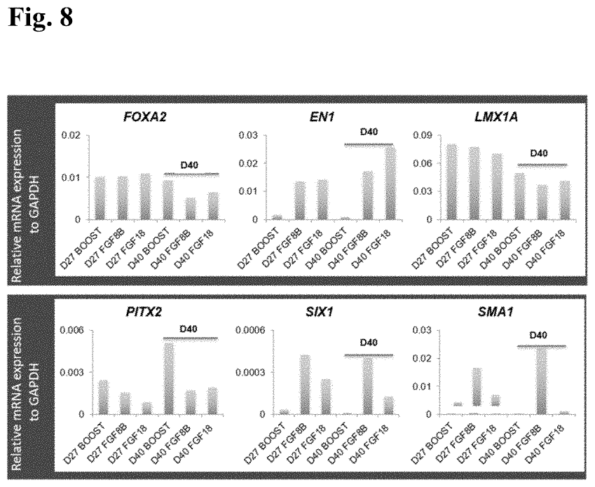

At the mature stage of differentiation, EN1 was still highly maintained in FGF8b and FGF18 treated conditions (see FIG. 8). Additionally, both FGF18 and FGF8b treated cells had reduced expression level of PITX2 as compared to the cells differentiated by the WNT-Boost protocol, while FGF18 treated cells had less expression levels of SMA1 and SIX1 than FGF8b treated cells (see FIG. 8). These results show that FGF18 treatment lead to sustained EN1 expression while minimizing or reducing the expression levels of non-mDA markers as compared to FGF8b treatment.

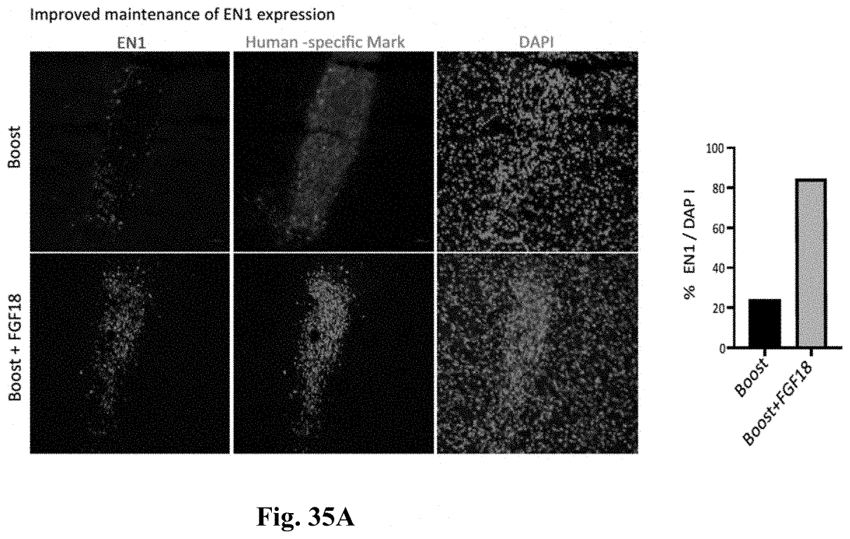

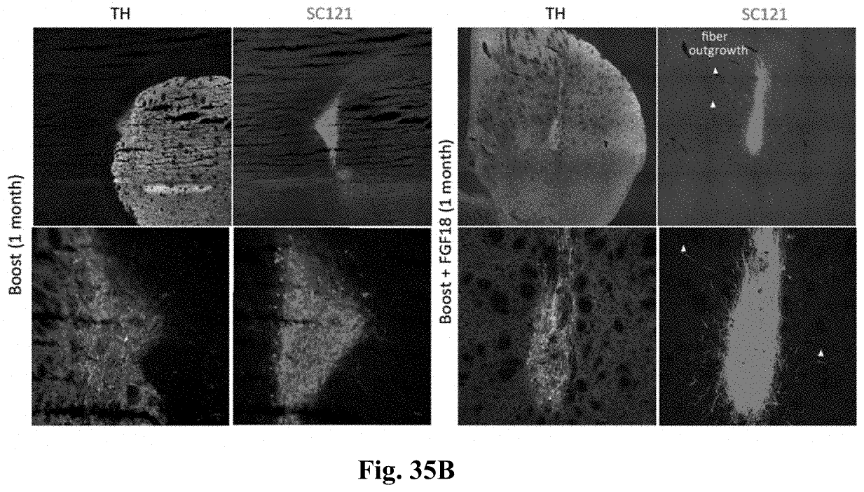

The in vivo survival of differentiated cells generated from WNT-boost+FGF18 protocol was examined. Cells generated from WNT-boost and WNT-boost+FGF18 protocols were grafted into intact mouse protein. Cells generated from WNT-boost+FGF18 protocol had improved maintenance of EN1 expression in vivo as compared to cells generated from WNT-boost protocol (FIG. 35A). Cells generated from WNT-boost+FGF18 protocol also had better striatal innervation with fibers already emerging from graft core towards periphery by 1 month post grafting (FIG. 35B).

### Example 2: Purification of mDAs Using a Reporter

NURR1 is a marker for post-mitotic and immature midbrain DA neurons, and also express in mature midbrain DA neurons. It is a transcription factor and contributes to DA differentiation and maintenance.

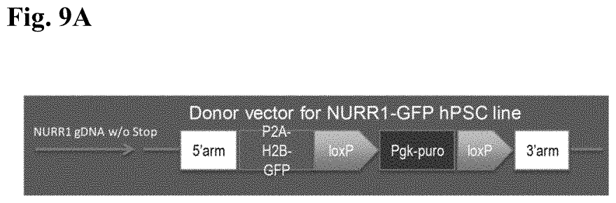

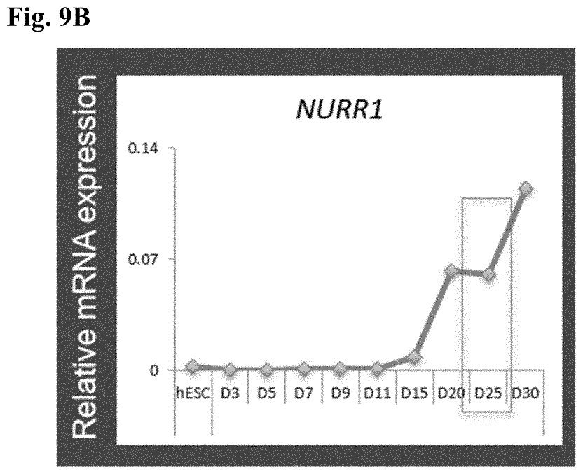

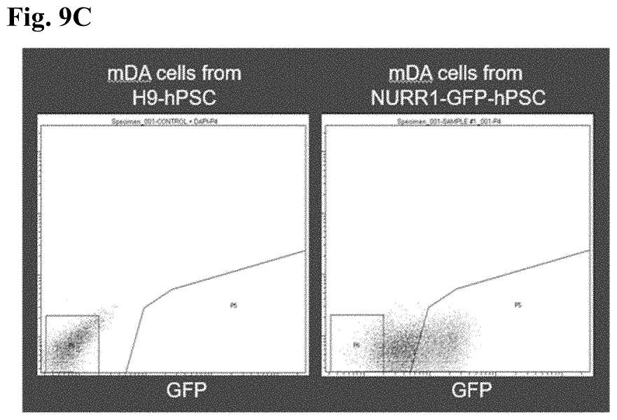

To purify mDAs from a cell population, the present example used an endogenous NURR1::GFP reporter hPSC (see FIG. 9A). mDAs were differentiated from the reporter cell line. FACS-based isolation of GFP positive cells was performed on day 25 of differentiation on the basis that NURR1 mRNA expression was highly induced from day 20 of differentiation (see FIGS. 9B-9C).

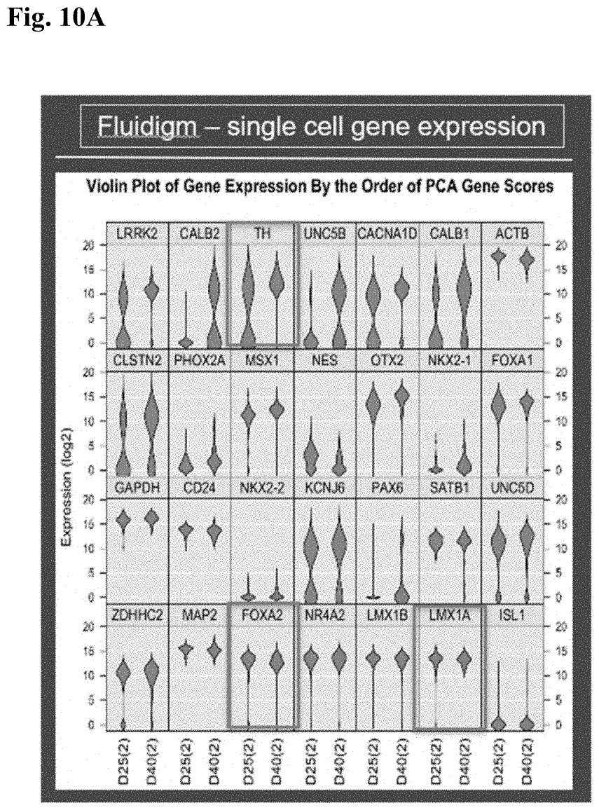

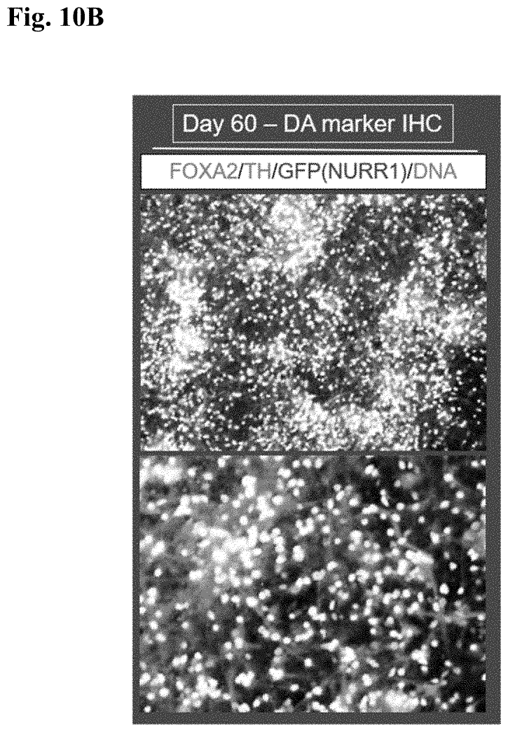

Single cell qRT-PCR was performed in NURR1:GFP positive cells isolate on day 25 and day 40 of differentiation. It was found that nearly about 100% NURR1:GFP positive cells expressed TH (a mature mDA marker), FOXA2 and LMX1A on day 40 of differentiation, indicating mDA fate (see FIG. 10A). Continuously culturing the isolated NURR1:GFP positive cells until day 60 showed that these cells expressed high level of TH, indicating that these cells were highly pure mDAs (see FIG. 10B).

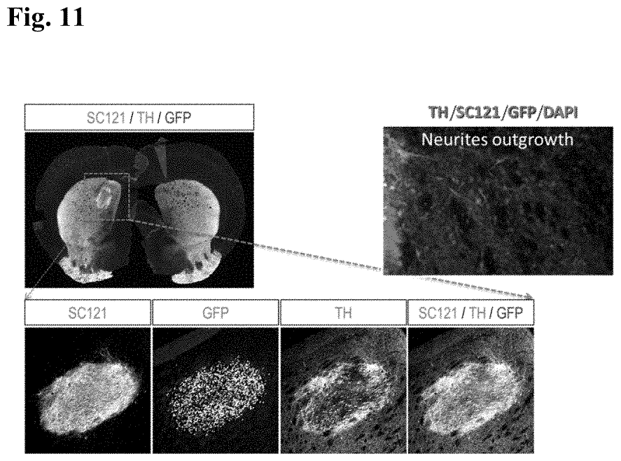

NURR1::GFP positive midbrain DA neurons on day 25 of differentiation were transplanted to nude mice. FIG. 11 shows that the transplanted cells survived in vivo, and expressed TH, human marker SC121, and GFP. Neurite outgrowth was found at the cell grafted region (see FIGS. 11).

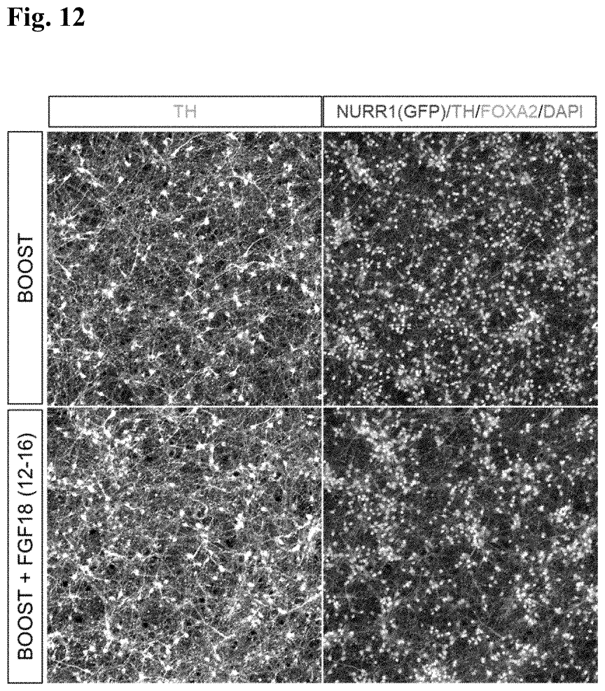

NURR1:GFP hPSCs were then cultured under WNT-Boost and WNT-boost+FGF18 (day 12-day 16) protocols. NURR1:GFP positive cells were isolated on day 25 of differentiation followed by continuous culturing until day 40. On day 40 of differentiation, these midbrain DA neurons expressed high TH along with FOXA2 (see FIG. 12).

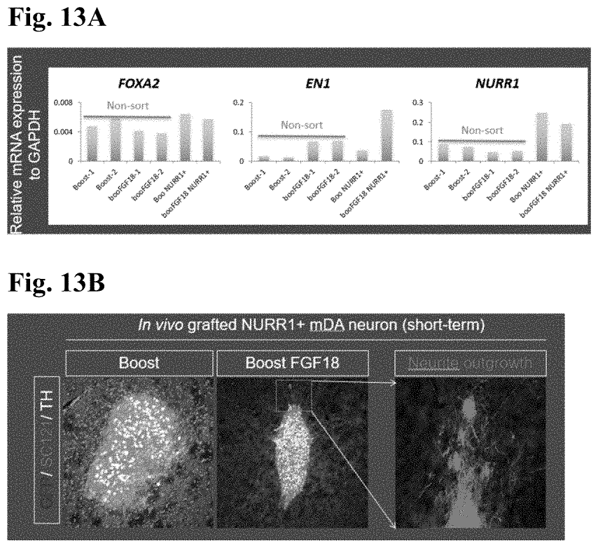

mRNA expression analysis shows that mDAs derived from WNT-Boost+FGF18 (day 12-day 16) protocol and sorted by NURR1:GFP had higher expression level of EN1 than mDAs derived from WNT-Boost and sorted by NURR1:GFP (see FIG. 13A). These sorted cells were transplanted to immuno-deficient mice. Both mDAs, derived from the WNT-Boost protocol and the WNT-Boost+FGF18 (day 12-day 16) protocol, showed excellent cell survival, and expressed markers SC121 and TH. However, FGF18 treated cells (WNT-Boost+FGF18 protocol) showed better neurite outgrowth from grafted region (see FIG. 13B).

### Example 3: Discovering Surface Markers in Purified DA Neurons

Published paper showed that each iPSC line has variation for the specific cell type induction. It is difficult to generate a reported line for each iPSC line used for purification of midbrain DA cells. Also genetically engineered cells are not suitable for clinical use. The present example identified candidate surface markers using the NURR1::GFP reporter line, in particular, surface markers enriched in NURR1::GFP positive cells but not NURR1::GFP negative cells, or vice versa.

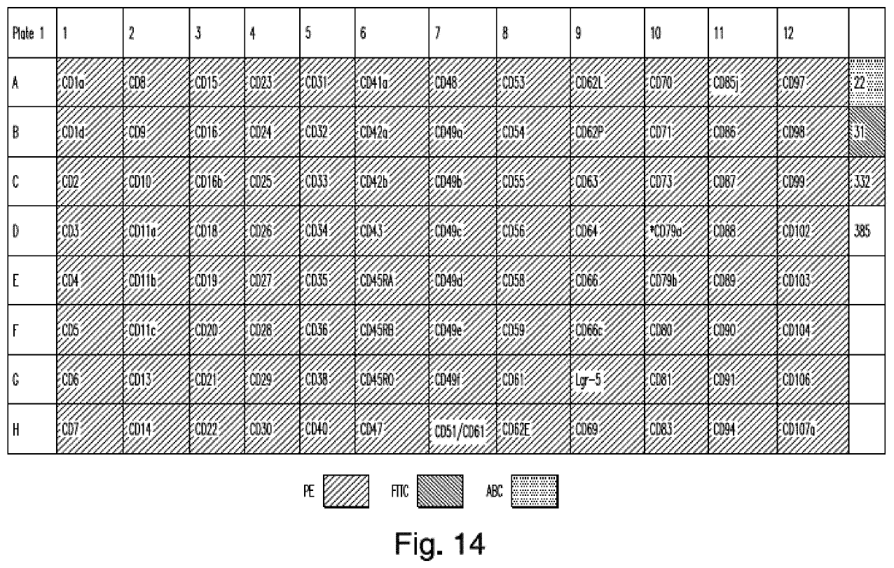

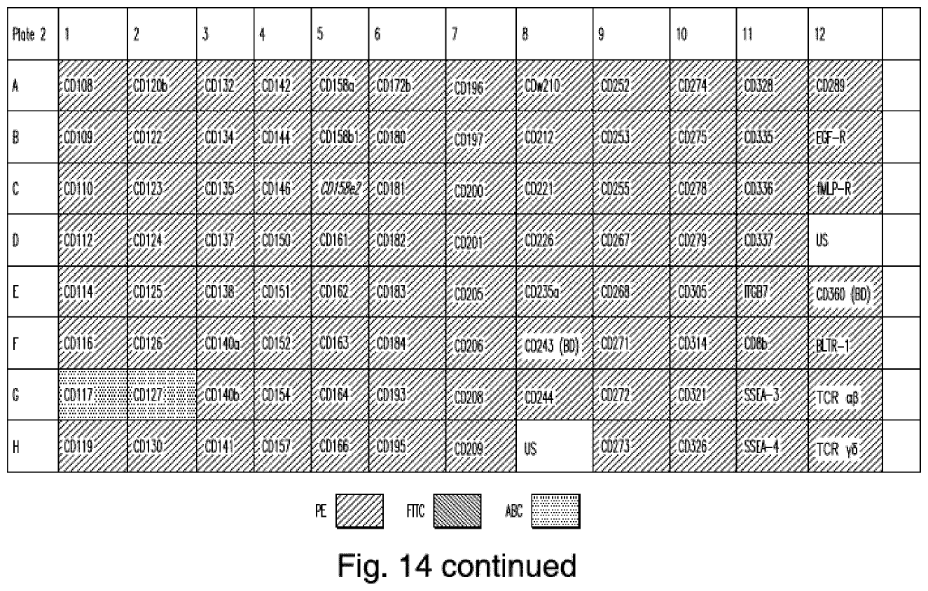

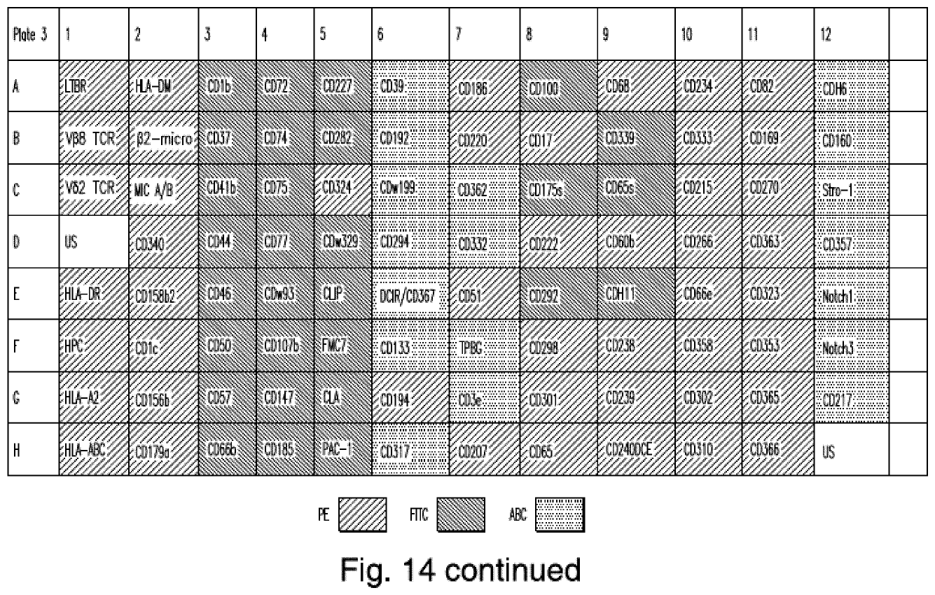

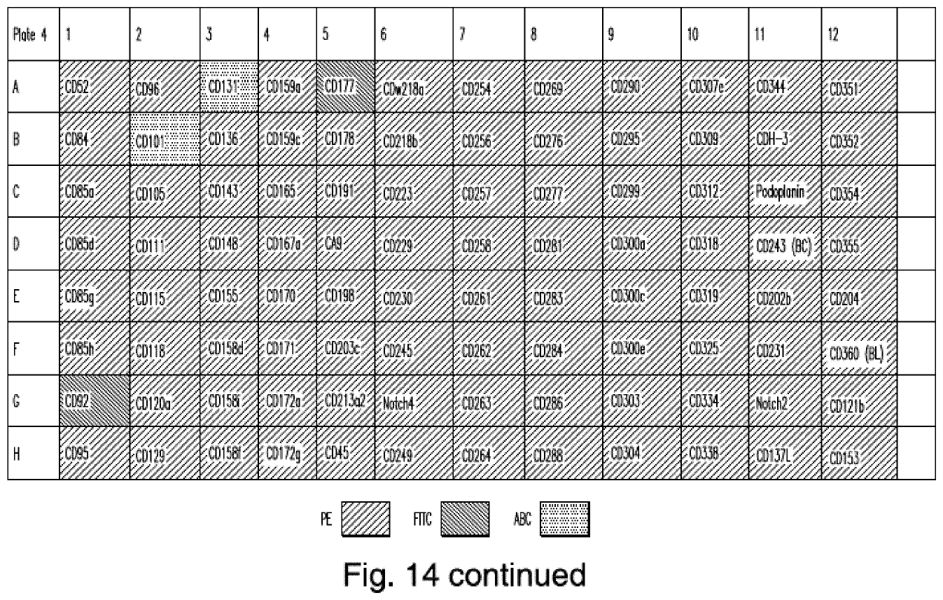

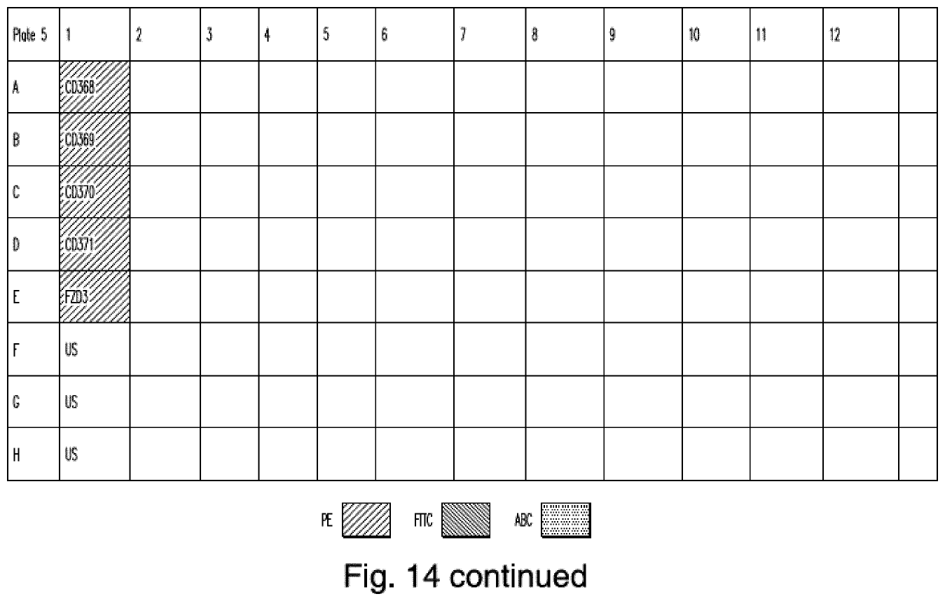

The present example tested 387 surface markers in mDA differentiated cells on day 25 of differentiation derived from the NURR1:GFP hPSC (see FIG. 14).

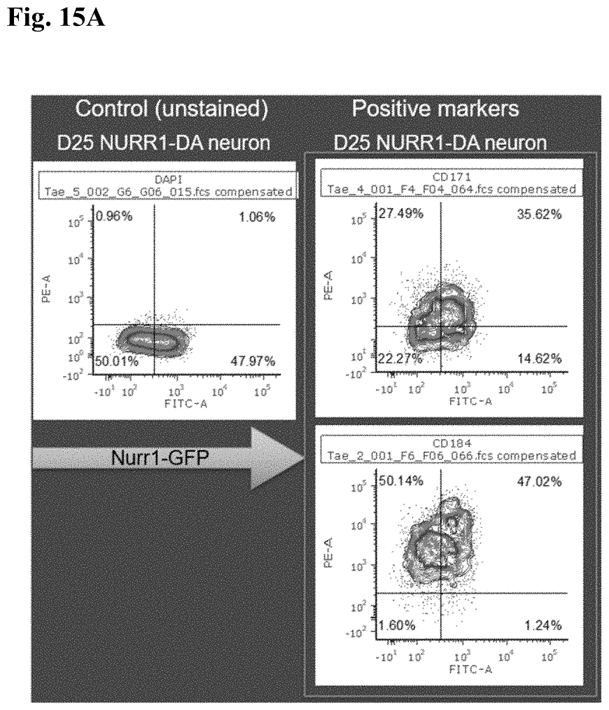

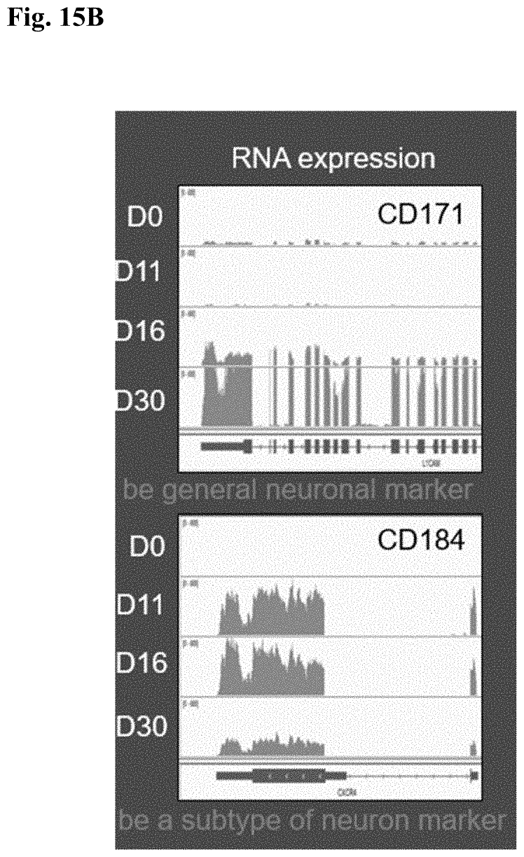

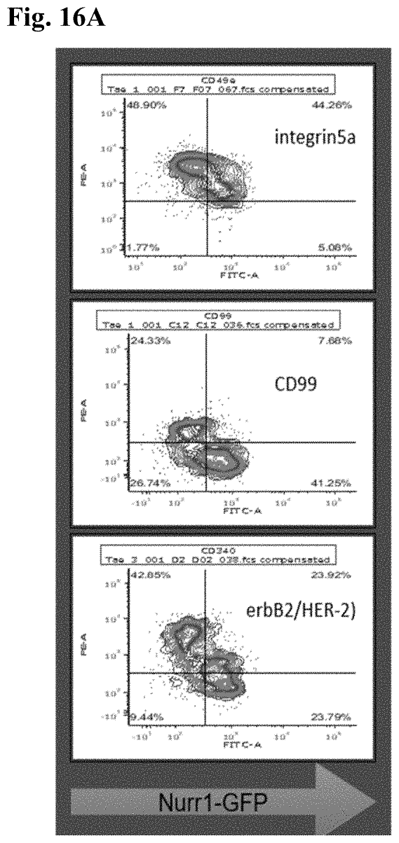

Two positive CD markers CD171 and CD184 were enriched in the NURR1::GFP positive population (see FIGS. 15A-15B), and 3 negative CD markers CD49e, 99 and 340 were enriched in the NURR1::GFP negative population (see FIGS. 16A-16B).

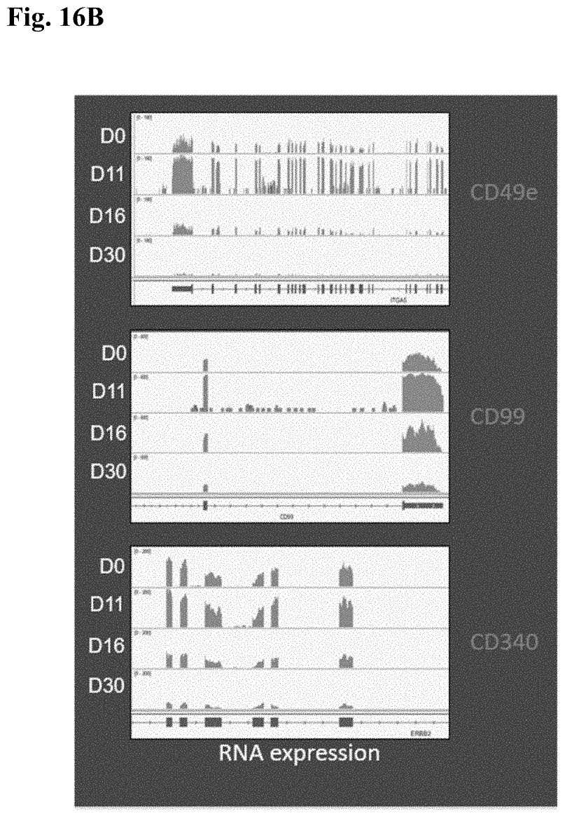

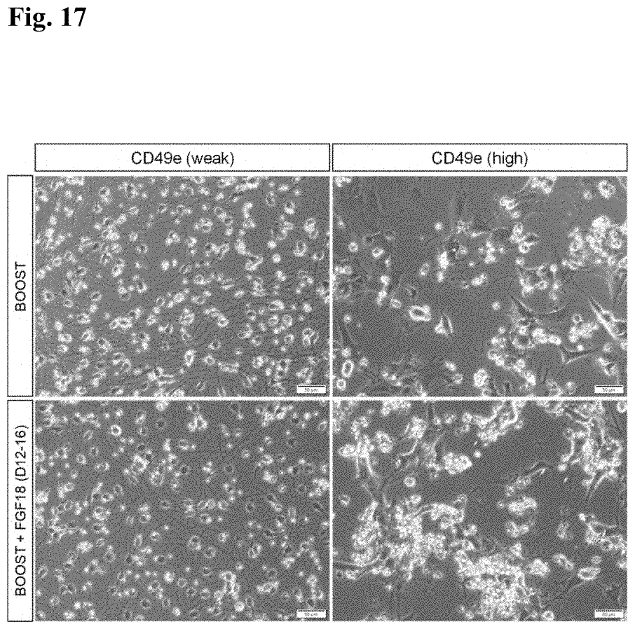

Cells were sorting by CD49e (Negative and/or weak expression) on day 25 of differentiation under the WNT-Boost protocol or the WNT-boost+FGF18 (day 12-day 16) protocol and were cultured for another 10 days. Cell morphology showed that these cells were substantially pure mDAs (see FIG. 17). The sorted mDAs on day 40 of differentiation (sorted on day 25, and further cultured for another 15 days) had high TH immune-staining (FIG. 18).

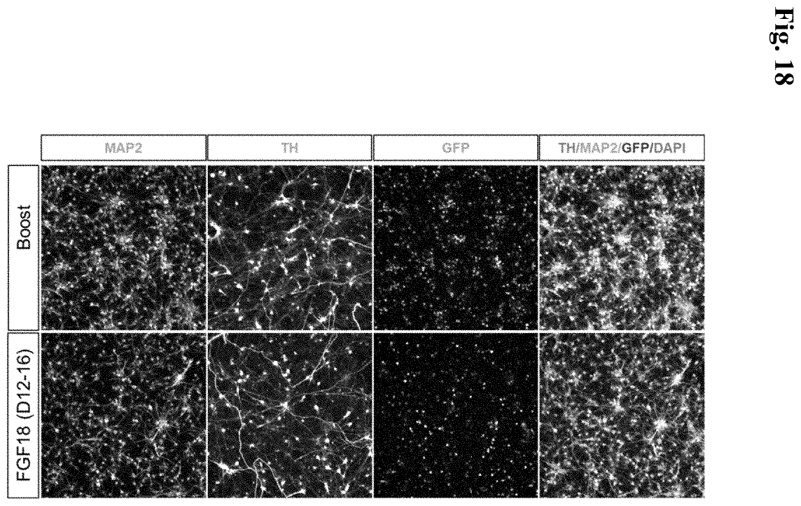

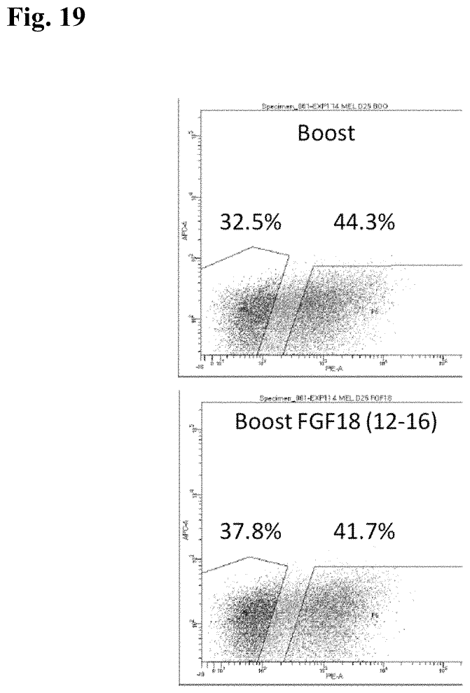

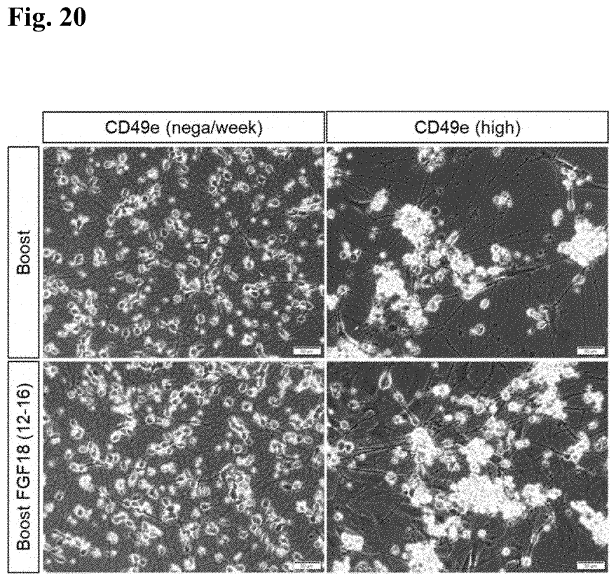

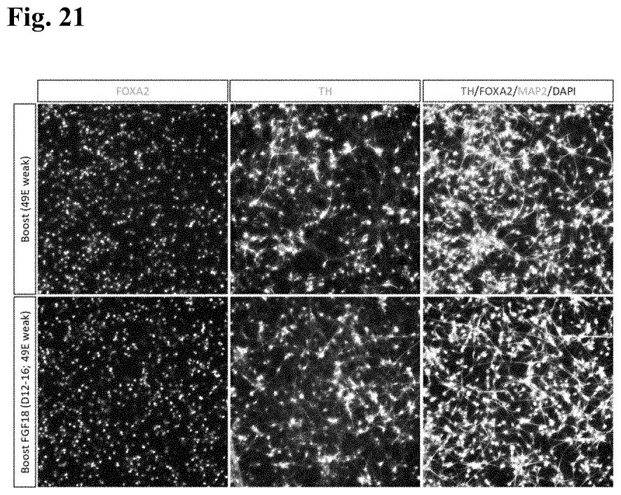

CD49e marker was tested in another hPSC line MEL1 for purifying midbrain DA neuron. Substantially pure mDA morphology was discovered in cells sorted with CD49e (Negative and/or weak expression) on day 25 of differentiation and continuously cultured for another 15 days (day 40 of differentiation) under the WNT-boost protocol and the WNT-boost+FGF18 (day 12-day 16) protocol (see FIGS. 19-20). These sorted mDAs had high TH immune-staining (see FIG. 21).

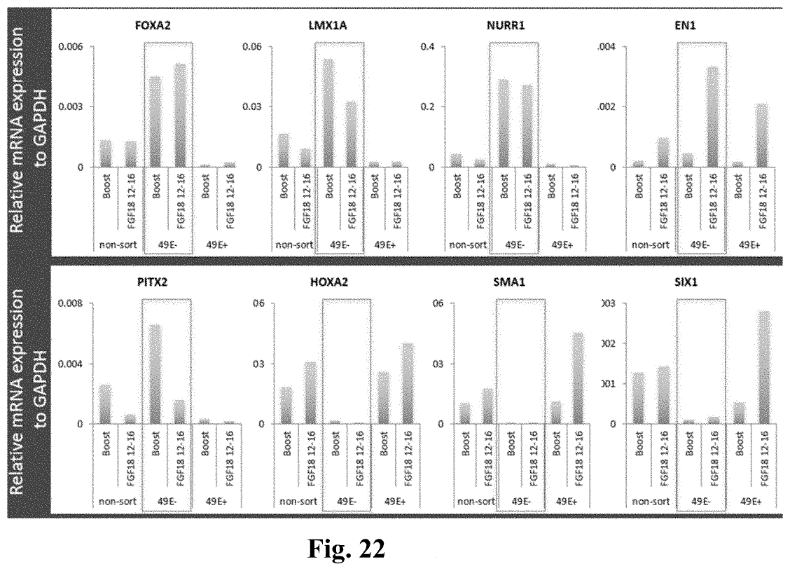

mRNA expression showed that CD49e sorted cells differentiated under the WNT-boost+FGF18 (day 12-day 16) protocol had higher expression level of EN1 and lower expression level of PITX2 (a glutamergic neuron (subtalamic necleous marker) than CD49e sorted cells differentiated under the WNT-Boost protocol. Sorted cells differentiated under both protocols had little or no expression level of non-midbrain DA markers (HOXA2, SMA1, and SIX1) (see FIG. 22).

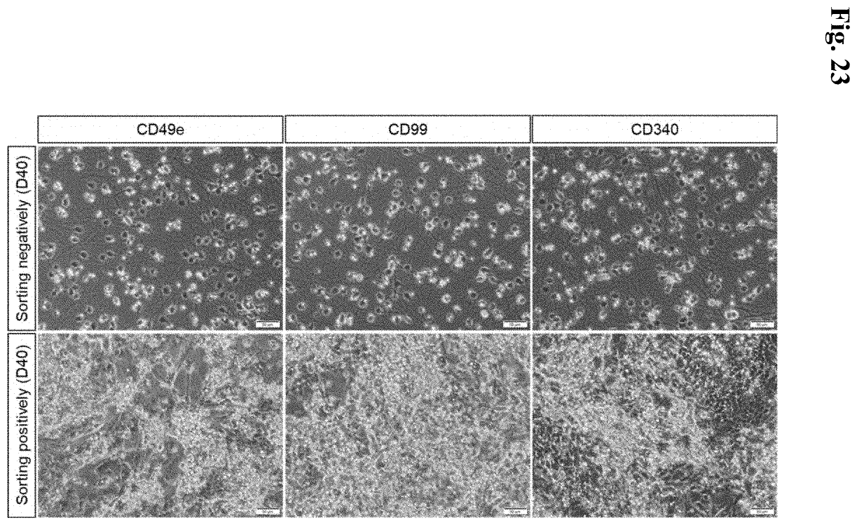

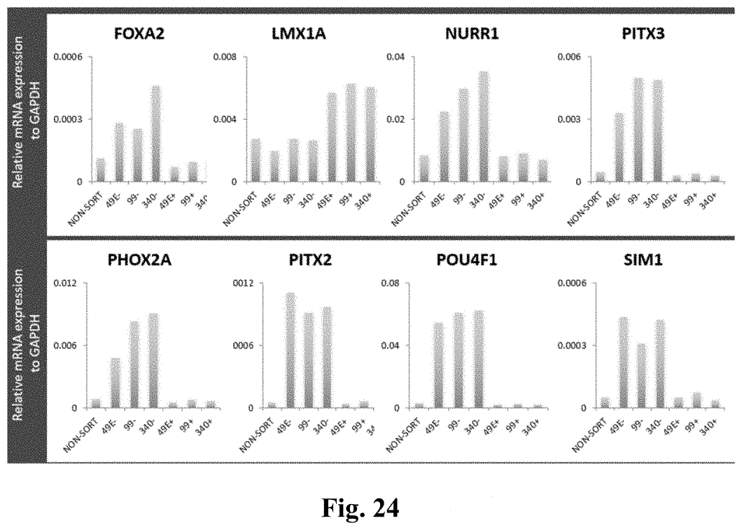

All three negative CD markers CD49e, CD99, and CD340 were tested. Substantially pure neuron shape was observed in cells sorted with CD49e, CD99, or CD340 (Negative and/or weak expressed cells) on day 25 of differentiation and continuously cultured for another 15 days (day 40 of differentiation) (see FIG. 23). However, mRNA expressions of the sorted cells showed increased expression of non-DA neuron markers, including PHOX2A, PITX2, POU4F1, and SIM1, suggesting that CD49e, CD99, or CD340 based isolation did not exclude non-DA neurons (FIG. 24).

It was considered that double sorting strategy-using CD184 might cure the deficiency of negative CD markers. Cxcr4 (CD184) is important for migration and orientation of midbrain DA neuron during mouse midbrain development. FGF18 treated mDAs can enrich A9 midbrain DA subtype after sorting with CD184⁺/CD49e⁻.

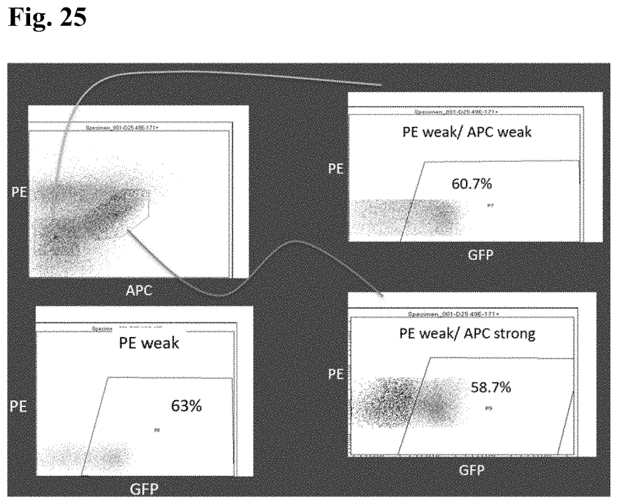

FACS sorting was performed with midbrain DA on day 25 of differentiation derived from NURR1::GFP hPSC using CD49E (PE) and CD171 (APC). It was found that single CD49e negatively sorted cells had about 63% NURR1:GFP portion. And CD171 positive sorting cannot enrich NURR1:GFP population combined with CD49e (see FIG. 25).

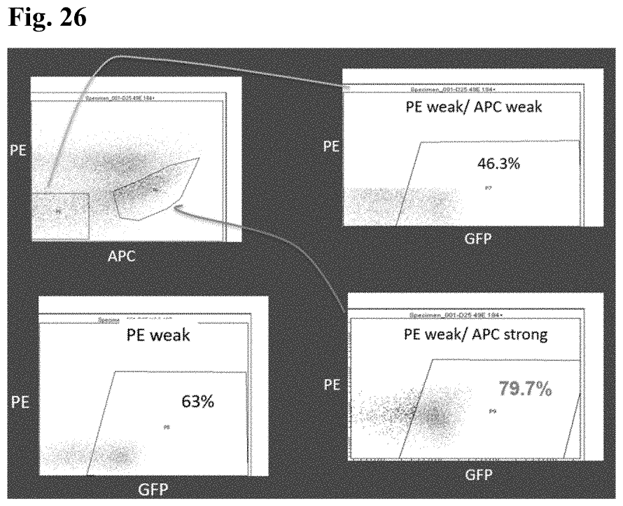

However, sorting with CD184 (positive expressed cells) with CD49e can enrich the NURR1:GFP portion to about 80% than single sorted cells (CD49e; 63%) (see FIG. 26).

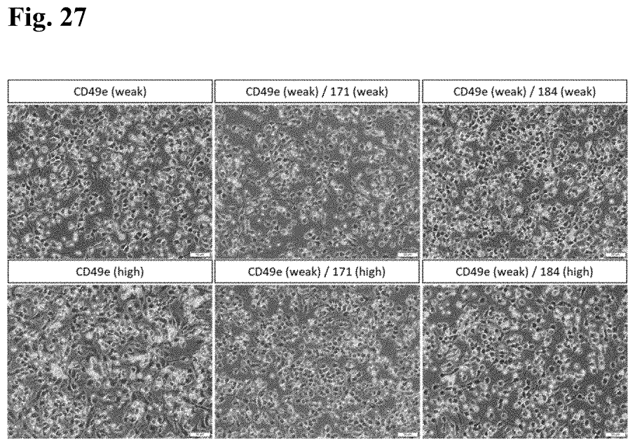

FACS sorted was then performed in cells on day 25 of differentiation by CD49e and CD171 or CD49e and CD184. Morphology of cells cultured 2 days after the sorting showed pure neuron shape except higher CD49e based sorted cells (see FIG. 27).

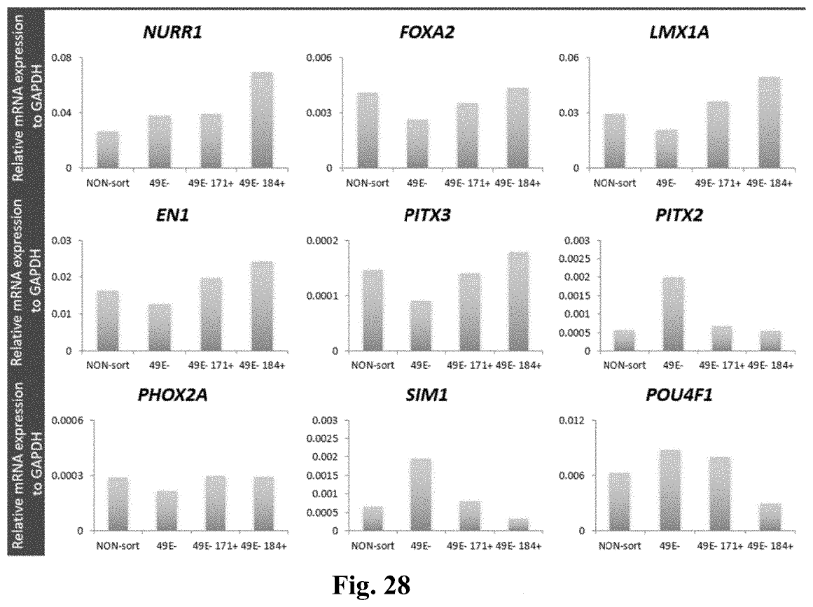

mRNA expression showed CD49e⁻/CD184⁺ (CD49e negative/CD184 positive) cells had higher expression level of mDA markers (NURR1, EN1, PITX3) and less expression level of non-mDA markers (PITX2, SIM1, and POU4F1) than cells sorted other ways (see FIG. 28).

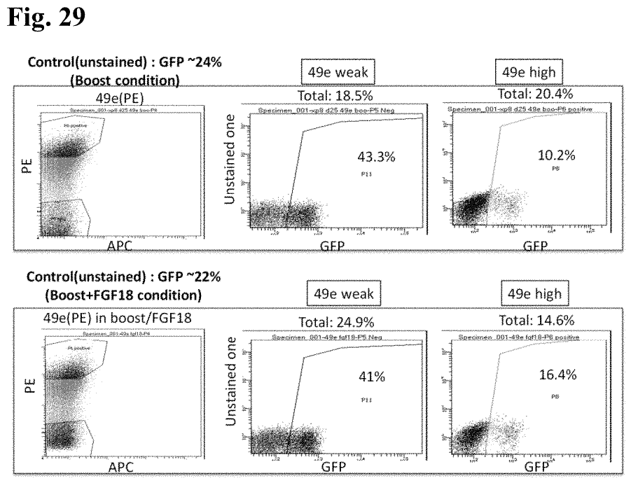

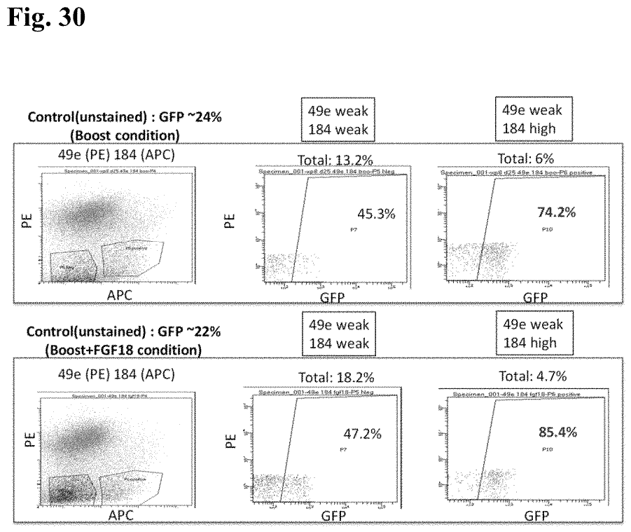

Next, it was investigated whether CD49e and CD184 could robustly sort mDAs. As shown in FIG. 29, single CD49e⁻ sorting enriched the NURR1:GFP positive population from ˜20% to up to 43% in in vitro differentiated cells. As shown in FIG. 30, double CD49e⁻ and CD184⁺ sorting enriched the NURR1::GFP positive portion to 74% and 85% in in vitro differentiated cells.

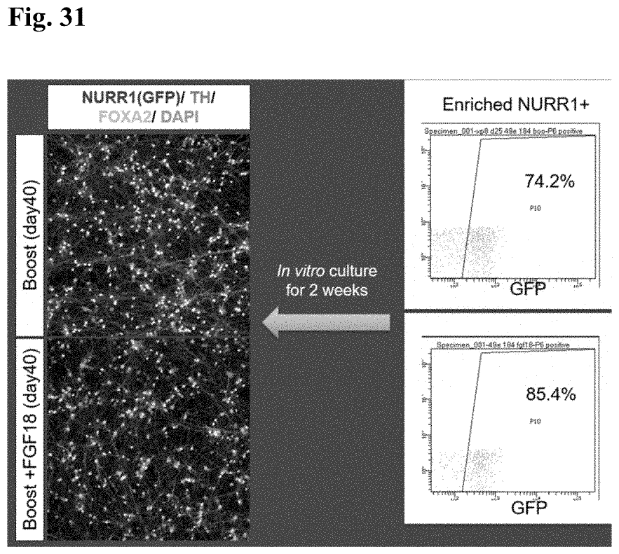

Followed by in vitro culturing for 2 week, sorted cells showed high TH⁺ mDAs co-expressing FOXA2 and GFP, which confirms mDA identity. (see FIG. 31).

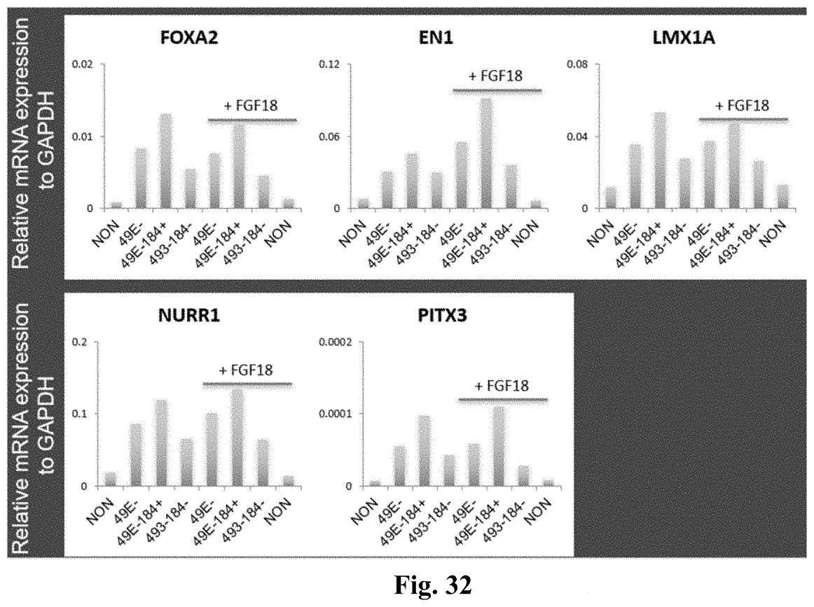

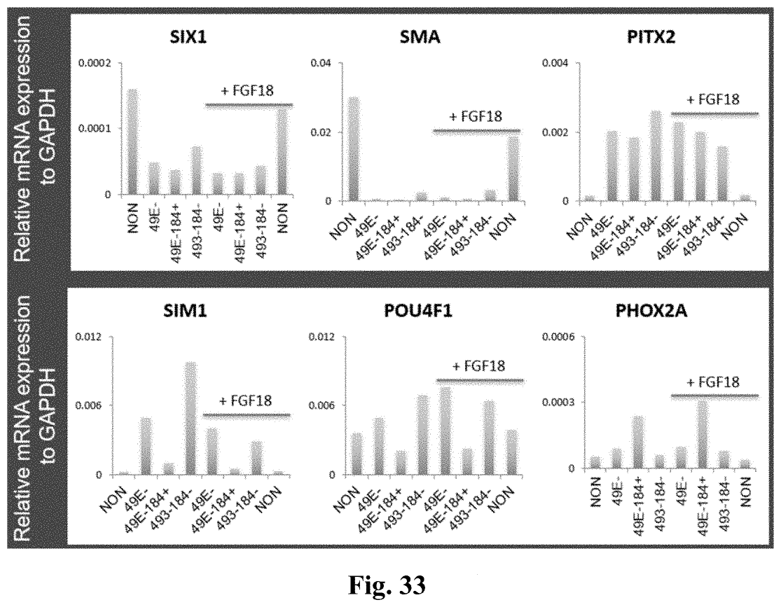

mRNA expression showed double CD marker-mediated sorted cells (CD49e− and CD184+) had generally higher expression of mDA markers (see FIG. 32) while less expression level of non-mDA marker (see FIG. 33) at day 40 of differentiation sorted by CD markers at day 25 than other CD sorted cells.

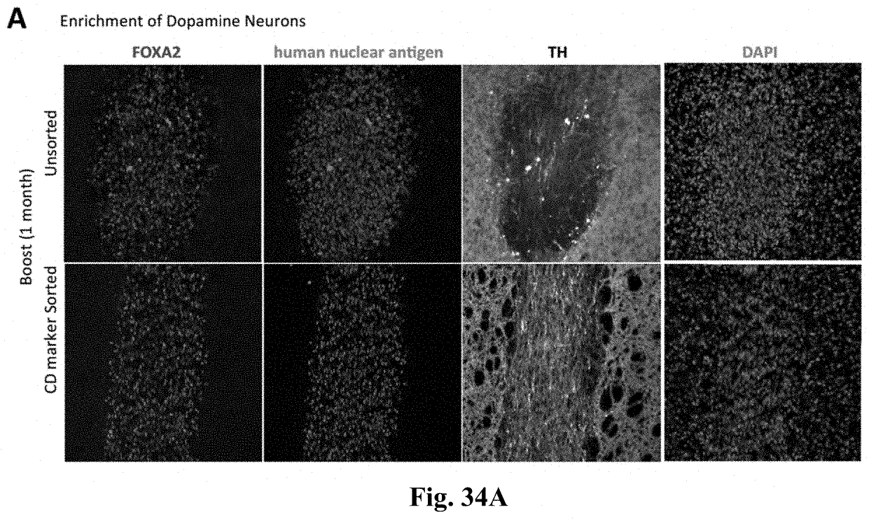

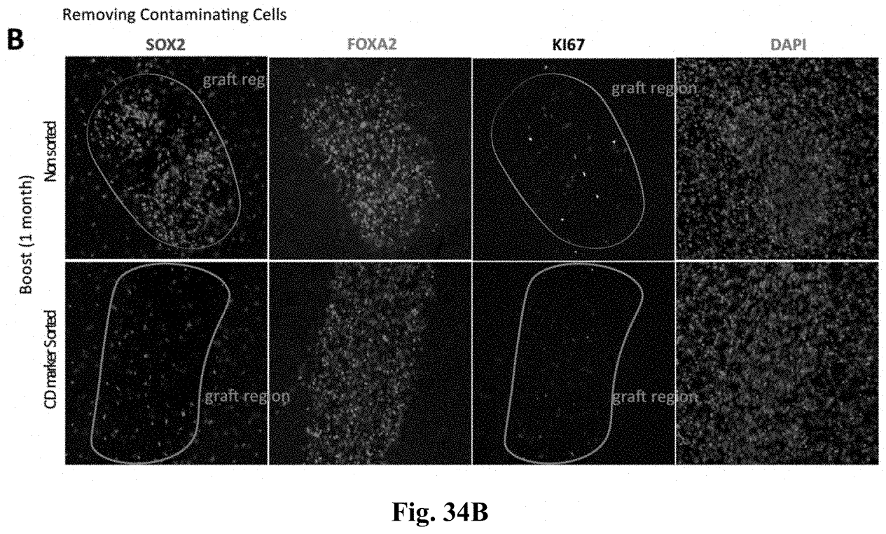

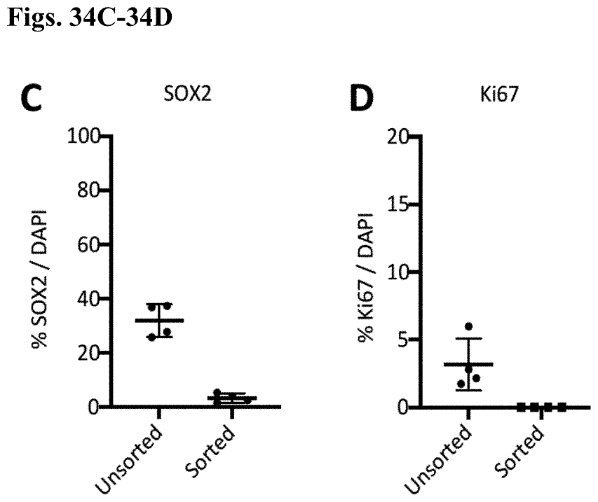

The in vivo survival of differentiated cells sorted with the presently disclosed novel surface markers was examined. Differentiated cells were generated from WNT-boost protocol, and CD49e weak CD184 high cells were sorted out and were grafted into intact mouse brains. Comparing to unsorted cells, tissues grafted with sorted cells had an enrichment of Th+ cells (FIG. 34A) and had reduced number of SOX2+ precursors and KI67+ dividing cells one month after grafting (FIGS. 34B-34D).

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the present disclosures of which are incorporated herein by reference in their entireties for all purposes

What is claimed is:

1. An in vitro method for inducing differentiation of stem cells, comprising:
contacting the stem cells with at least one inhibitor of Small Mothers Against Decapentaplegic (SMAD) signaling, at least one activator of Sonic hedgehog (SHH) signaling, and at least one activator of wingless (Wnt) signaling; and contacting the cells with at least one activator of fibroblast growth factor (FGF) signaling to obtain a population of differentiated cells expressing at least one marker indicating a midbrain dopamine neuron (mDA) or a precursor thereof, wherein:
(i) the at least one activator of FGF signaling is selected from the group consisting of FGF18, FGF17, FGF8a, and combination thereof, and/or
(ii) the initial contact of the cells with the at least one activator of FGF signaling is at least about 5 days from the initial contact of the cells with the at least one inhibitor of SMAD signaling.

2. The method of claim 1, wherein the cells are contacted with the at least one activator of FGF signaling for (i) at least about 1 days, (ii) up to about 15 days, and/or (iii) about 5 days.

3. The method of claim 1, wherein the initial contact of the cells with the at least one activator of FGF signaling is:
(i) at least about 5 days from the initial contact of the cells with the at least one inhibitor of SMAD signaling,
(ii) about 10 days from the initial contact of the cells with the at least one inhibitor of SMAD signaling, or
(iii) 12 days from the initial contact of the cells with the at least one inhibitor of SMAD signaling.

4. The method of claim 1, wherein the cells are contacted with the at least one inhibitor of SMAD signaling for (i) about 5 days, or (ii) for 7 days.

5. The method of claim 1, wherein the cells are contacted with the at least one activator of SHH signaling for (i) about 5 days, or (ii) for 7 days.

6. The method of claim 1, wherein the cells are contacted with the at least one activator of Wnt signaling for about 10 days, or (ii) for 12 days.

7. The method of claim 1, wherein the concentration of the at least one activator of Wnt signaling is
(i) increased about 4 days from its initial contact with the stem cells;
(ii) increased by between about 300% and about 1000% from the initial concentration of the at least one activator of Wnt signaling;
(iii) increased to a concentration of between about 3 μM and about 10 μM;
(iv) increased to a concentration of about 3 μM; and/or
(v) increased to a concentration of about 7.5 μM.

8. The method of claim 1, wherein
(i) the at least one activator of FGF signaling comprises FGF18;
(ii) the at least one inhibitor of SMAD signaling is selected from the group consisting of inhibitors of TGFβ/Activin-Nodal signaling, inhibitors of bone morphogenetic protein (BMP) signaling, and combinations thereof;
(iii) the at least one activator of Wnt signaling comprises an inhibitor of glycogen synthase kinase 3β (GSK3β) signaling;
(iv) the at least one activator of Wnt signaling is selected from the group consisting of CHIR99021, Wnt3A, Wnt1, derivatives thereof, and mixtures thereof, and/or
(v) the at least one activator of SHH signaling is selected from the group consisting of SHH protein, Smoothened agonists (SAG), derivatives thereof, and mixtures thereof.

9. The method of claim 8, wherein
(i) the at least one inhibitor of TGFβ/Activin-Nodal signaling comprises an inhibitor of ALK5;
(ii) the at least one inhibitor of TGFβ/Activin-Nodal signaling comprises SB431542, or a derivative, or a mixture thereof,
(iii) the at least one inhibitor of TGFβ/Activin-Nodal signaling comprises SB431542;
(iv) the at least one inhibitor of BMP signaling comprises LDN193189, Noggin, dorsomorphin, a derivative thereof, or a mixture thereof,
(v) the at least one inhibitor of BMP comprises LDN-193189; or
(vi) the SHH protein comprises a recombinant SHH, a purified SHH, or a combination of the foregoing.

10. The method of claim 9, wherein the derivative of SB431542 is A83-01.

11. The method of claim 10, wherein the recombinant SHH comprises i) a recombinant protein that is at least about 80% identical to a mouse Sonic Hedgehog N-terminal fragment, or ii) the recombinant SHH comprises SHH C25II.

12. The method of claim 10, wherein the SAG comprises purmorphamine.

13. The method of claim 1, wherein the at least one marker indicating a midbrain dopamine neuron or a precursor thereof is selected from the group consisting of EN1, OTX2, TH, NURR1, FOXA2, PITX3, LMX1A, LMO3, SNCA, ADCAP1, CHRNA4, GIRK2, and combinations thereof.

14. The method of claim 1, wherein the differentiated cells have a detectable level of expression of
(i) the at least one marker indicating a midbrain dopamine neuron or a precursor thereof at least about 10 days from the initial contact of the stem cells with the at least one inhibitor of SMAD signaling;
(ii) EN1 about 30 days from the initial contact of the stem cells with the at least one inhibitor of SMAD signaling; or
(iii) EN1 about 40 days from the initial contact of the stem cells with the at least one inhibitor of SMAD signaling.

15. The method of claim 1, wherein the differentiated cells do not express at least one marker selected from the group consisting of PAX6, EMX2, LHX2, SMA, SIX1, PITX2, SIM1, POU4F1, PHOX2A, BARHL1, BARHL2, GBX2, HOXA2, HOXB2, POU5F1, NANOG, and combinations thereof.

16. The method of claim 1, further comprising subjecting the population of differentiated cells to conditions favoring differentiation of midbrain dopamine neuron precursors to midbrain dopamine neurons.

17. The method of claim 16, wherein the conditions comprise exposing the cells to at least one of brain-derived neurotrophic factor (BDNF), glial cell-derived neurotrophic factor (GDNF), Cyclic adenosine monophosphate (cAMP), Transforming growth factor beta 3 (TGFP3), ascorbic acid (AA), and DAPT.

18. The method of claim 1, wherein a) the stem cells are selected from the group consisting of human nonembryonic stem cells, nonhuman primate nonembryonic stem cells, rodent nonembryonic stem cells, human embryonic stem cells, nonhuman primate embryonic stem cells, rodent embryonic stem cells, human induced pluripotent stem cells, nonhuman primate induced pluripotent stem cells, rodent induced pluripotent stem cells, human recombinant pluripotent cells, nonhuman primate recombinant pluripotent cells, and rodent recombinant pluripotent cells; b) the stem cells are human stem cells, and/or c) the stem cells are pluripotent stem cells or multipotent stem cells.

19. The method of claim 18, wherein the pluripotent stem cells are selected from the group consisting of embryonic stem cells, induced pluripotent stem cells, and combinations thereof.

20. A cell population of in vitro differentiated cells, wherein said in vitro differentiated cells are obtained by a method of claim 1.

21. A cell population of in vitro differentiated cells, wherein at least about 50% of the cells express at least one marker indicating a midbrain dopamine neuron or a precursor thereof, and less than about 50% of the differentiated cells express at least one marker selected from the group consisting of PAX6, EMX2, LHX2, SMA, SIX1, PITX2, SIM1, POU4F1, PHOX2A, BARHL1, BARHL2, GBX2, HOXA2, HOXB2, POU5F1, NANOG, and combinations thereof.

22. A composition comprising the cell population of claim 21.

23. A method for isolating midbrain dopamine neurons and precursors thereof from a population of cells, comprising isolating cells that (a) do not express a detectable level or express a reduced level of at least one negative surface marker as compared to the mean expression level of the at least one negative surface marker in the population of cells; and (b) an increased level of at least one positive surface marker as compared to the mean expression level of the at least one positive marker in the population of cells.

24. A cell population of in vitro differentiated cells, wherein at least about 50% of the cells express an increased level of at least one positive surface marker as compared to the mean expression level of the at least one positive marker in the population of cells; and do not express a detectable level or express a reduced level of at least one negative surface marker as compared to the mean expression level of the at least one negative surface marker in the population of cells.

25. A composition comprising the cell population of claim 24.

26. A kit for inducing differentiation of stem cells to midbrain dopamine neurons or precursors thereof, comprising:
(a) at least one inhibitor of SMAD signaling;
(b) at least one activator of SHH signaling;
(c) at least one activator of Wnt signaling; and
(d) at least one activator of FGF signaling.

27. A method of preventing and/or treating a neurodegenerative disorder in a subject, comprising administering to the subject an effective amount of the cell population of claim 21.
